(19) United States
(12) Patent Application Publication
(10) Pub. No.: US 2022/0112542 A1
(43) Pub. Date: Apr. 14, 2022

(54) SIGNATURE FOR DIAGNOSIS OF BACTERIAL VS VIRAL INFECTIONS

(71) Applicants: THE BOARD OF TRUSTEES OF THE LELAND STANFORD JUNIOR UNIVERSITY, Stanford, CA (US); THE UNITED STATES GOVERNMENT AS REPRESENTED BY THE DEPARTMENT OF VETERANS AFFAIRS, Washington, DC (US)

(72) Inventors: Purvesh KHATRI, Menlo Park, CA (US); Aditya Manohar RAO, Stanford, CA (US); David A. RELMAN, Palo Alto, CA (US); Stephen POPPER, Stanford, CA (US); Timothy E. SWEENEY, Redwood City, CA (US)

(21) Appl. No.: 17/441,670

(22) PCT Filed: Feb. 14, 2020
(86) PCT No.: PCT/US2020/018414

§ 371 Date: Sep. 21, 2021

Related U.S. Application Data
(60) Provisional application No. 62/823,460, filed on Mar. 25, 2019.

(51) Int. Cl. C12Q 1/689; C12Q 1/686; C12Q 1/70; G06N 20/00

(52) U.S. Cl. CPC C12Q 1/689; C12Q 1/686; C12Q 2600/158; G06N 20/00; C12Q 1/70

(57) ABSTRACT

This disclosure provides a gene expression-based method for determining whether a subject has a viral infection or a bacterial infection. A kit for performing the method is also provided.

### CROSS-REFERENCING

This application claims the benefit of provisional application Ser. No. 62/823,460, filed on Mar. 25, 2019, which application is incorporated by reference herein in its entirety for all purposes.

### STATEMENT REGARDING FEDERALLY SPONSORED RESEARCH

This invention was made with Government support under contracts AI057229 and AI109662 awarded by the National Institutes of Health. The Government has certain rights in the invention.

### BACKGROUND

Early and accurate diagnosis of infection is key to improving patient outcomes and reducing antibiotic resistance. The mortality rate of bacterial sepsis increases 8% for each hour by which antibiotics are delayed; however, giving antibiotics to patients without bacterial infections increases rates of morbidity and antimicrobial resistance. The rate of inappropriate antibiotic prescriptions in the hospital setting is estimated at 30-50%, and would be aided by improved diagnostics. Strikingly, close to 95% of patients given antibiotics for suspected enteric fever have negative cultures. There is currently no gold-standard point of care diagnostic that can broadly determine the presence and type of infection. Thus, the White House has established a National Action Plan for Combating Antibiotic-Resistant Bacteria, which called for “point-of-need diagnostic tests to distinguish rapidly between bacterial and viral infections”.

While come PCR-based molecular diagnostics can profile pathogens directly from a blood culture, such methods rely on the presence of adequate numbers of pathogens in the blood. Moreover, they are limited to detecting a discrete range of pathogens. As a result, there is growing interest in molecular diagnostics that profile the host gene response. These include diagnostics that can distinguish the presence of infection as compared to inflamed but non-infected patients. Overall, while great promise has been shown in this field, no host gene expression infection diagnostic has yet made it into clinical practice.

There remains a need for sensitive and specific diagnostic tests that can distinguish between bacterial and viral infections.

### SUMMARY

Patients can be classified as having a viral infection or bacterial infection based on the expression of eight genes: by JUP, SUCLG2, IFI27, FCER1A, HESX1, SMARCD3, ICAM1, and EBI3. Increased JUP, SUCLG2, IFI27, FCER1A, HESX1 expression indicates that the subject has a viral infection and increased SMARCD3, ICAM1, EBI3 indicates that the subject has a bacterial infection.

In some embodiments a method of analyzing a sample is provided. This method may comprise: (a) obtaining a sample of RNA from a subject; and (b) measuring the amount of RNA transcripts encoded by JUP, SUCLG2, IFI27, FCER1A, HESX1, SMARCD3, ICAM1, and EBI3 in the sample, to produce gene expression data. This method may further comprise, based on the gene expression data, providing a report indicating whether the subject has a viral infection or a bacterial infection, wherein: (i) increased JUP, SUCLG2, IFI27, FCER1A, HESX1 expression indicates that the subject has a viral infection; and (ii) increased SMARCD3, ICAM1, EBI3 indicates that the subject has a bacterial infection.

In some embodiments, a method of treatment is provided. In these embodiments, the method may comprise (a) receiving a report indicating whether the subject has a viral infection or a bacterial infection, wherein the report is based on the gene expression data obtained by measuring the amount of RNA transcripts encoded by JUP, SUCLG2, IFI27, FCER1A, HESX1, SMARCD3, ICAM1, and EBI3, and (b) identifying the patient as having increased JUP, SUCLG2, IFI27, and FCER1A, and HESX1 expression, and treating the subject with anti-viral therapy; or (c) identifying the patient as having increased SMARCD3, ICAM1, EBI3 expression; and treating the subject with an anti-bacterial therapy.

Kits for performing the method are also provided.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 provides an overview of MANATEE.

FIG. 2 provides an 8-gene signature that distinguishes viral infections from intracellular and extracellular bacterial infections with high accuracy in the discovery and held-out validation data.

FIG. 3 provides an 8-gene signature that distinguishes viral infections from intracellular and extracellular bacterial infections with high accuracy in independent whole blood datasets.

FIG. 4 provides an 8-gene signature that distinguishes viral infections from intracellular and extracellular bacterial infections with high accuracy in independent PBMC datasets.

FIG. 5 provides an 8-gene signature that distinguishes viral infections from intracellular and extracellular bacterial infections with high accuracy in a prospectively enrolled cohort of patients with bacterial or viral infections in Nepal.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that, as used in this specification and the appended claims, the singular forms “a”, “an” and “the” include plural referents unless the content clearly dictates otherwise. Thus, for example, reference to “an agonist” includes a mixture of two or more such agonists, and the like.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As noted above, a method of analyzing a sample is provided. In some embodiments the method comprises (a) obtaining a sample of RNA from a subject; and (b) measuring the amount of RNA transcripts encoded by JUP, SUCLG2, IFI27, FCER1A, HESX1, SMARCD3, ICAM1, and EBI3 in the sample, to produce gene expression data. The method may be used in a variety of diagnostic and therapeutic methods, as described below.

Diagnostic Methods

As noted above, the method may be used to determine if a subject has a viral infection or bacterial infection. In some embodiments, the method may comprise: (a) obtaining a sample of RNA from a subject; (b) measuring the amount of RNA transcripts encoded by JUP, SUCLG2, IFI27, FCER1A, HESX1, SMARCD3, ICAM1, and EBI3 in the sample, to produce gene expression data and (c) providing a report indicating whether the subject has a viral infection or a bacterial infection, wherein: (i) increased JUP, SUCLG2, IFI27, FCER1A, HESX1 expression indicates that the subject has a viral infection; and (ii) increased SMARCD3, ICAM1, EBI3 indicates that the subject has a bacterial infection.

The measuring step can be done using any suitable method. For example, the amount of the RNA transcripts in the sample may be measured by RNA-seq (see, e.g., Morin et al BioTechniques 2008 45: 81-94; Wang et al 2009 Nature Reviews Genetics 10: 57-63), RT-PCR (Freeman et al BioTechniques 1999 26: 112-22, 124-5), or by labeling the RNA or cDNA made from the same and hybridizing the labeled RNA or cDNA to an array. An array may contain spatially-addressable or optically-addressable sequence-specific oligonucleotide probes that specifically hybridize to transcripts being measured, or cDNA made from the same. Spatially-addressable arrays (which are commonly referred to as “microarrays” in the art) are described in, e.g., Sealfon et al (see, e.g., Methods Mol Biol. 2011; 671:3-34). Optically-addressable arrays (which are commonly referred to as “bead arrays” in the art) use beads that internally dyed with fluorophores of differing colors, intensities and/or ratios such that the beads can be distinguished from each other, where the beads are also attached to an oligonucleotide probe. Exemplary bead-based assays are described in Dupont et al (J. Reprod Immunol. 2005 66:175-91) and Khalifian et al (J Invest Dermatol. 2015 135: 1-5). The abundance of transcripts in a sample can also be analyzed by quantitative RT-PCR or isothermal amplification method such as those described in Gao et al (J. Virol Methods. 2018 255: 71-75), Pease et al (Biomed Microdevices (2018) 20: 56) or Nixon et (Biomol. Det. and Quant 2014 2: 4-10), for example. Many other methods for measuring the amount of an RNA transcript in a sample are known in the art.

The sample of RNA obtained from the subject may comprise RNA isolated from whole blood, white blood cells, peripheral blood mononuclear cells (PBMC), neutrophils or buffy coat, for example. Methods for making total RNA, polyA+ RNA, RNA that has been depleted for abundant transcripts, and RNA that has been enriched for the transcripts being measured are well known (see, e.g., Hitchen et al J Biomol Tech. 2013 24: S43-S44). If the method involves making cDNA from the RNA, then the cDNA may be made using an oligo(d)T primer, a random primer or a population of gene-specific primers that hybridize to the transcripts being analyzed.

In measuring the transcript, the absolute amount of each transcript may be determined, or the amount of each transcript relative to one or more control transcript may be determined. Whether the amount of a transcript is increased or decreased may be in relation to the amount of the transcript (e.g., the average amount of the transcript) in control samples (e.g., in blood samples collected from a population of at least 100, at least 200, or at least 500 subjects that are known or not known to have viral and/or bacterial infections).

In some embodiments, the method may comprise providing a report indicating whether the subject has a viral or bacterial infection based on the measurements of the amounts of the transcripts. In some embodiments, this step may involve calculating a score based on the weighted amounts of each of the transcripts, where the scores correlates with the phenotype and can be a number such as a probability, likelihood or score out of 10, for example. In these embodiments, the method may comprise inputting the amounts of each of the transcripts into one or more algorithms, executing the algorithms, and receiving a score for each phenotype based on the calculations. In these embodiments, other measurements from the subject, e.g., whether the subject is male, the age of the subject, white blood cell count, neutrophils count, band count, lymphocyte count, monocyte count, whether the subject is immunosuppressed, and/or whether there are Gram-negative bacteria present, etc., may be input into the algorithm.

In some embodiments, the method may involve creating the report e.g., in an electronic form, and forwarding the report to a doctor or other medical professional to help identify a suitable course of action, e.g., to identify a suitable therapy for the subject. The report may be used along with other metrics as a diagnostic to determine whether the subject has a viral of bacterial infection.

In any embodiment, report can be forwarded to a “remote location”, where “remote location,” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like. In certain embodiments, the report may be analyzed by an MD or other qualified medical professional, and a report based on the results of the analysis of the image may be forwarded to the subject from which the sample was obtained.

In computer-related embodiments, a system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining whether the subject has a viral or bacterial infection using the measurements described above as inputs. The computer processor is coupled to the storage component and configured to execute the instructions stored in the storage component in order to receive patient data and analyze patient data according to one or more algorithms. The display component may display information regarding the diagnosis of the patient.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

Therapeutic Methods

Therapeutic methods are also provided. In some embodiments, these methods may comprise identifying a subject as having a viral infection or a bacterial infection using the methods described above, and treating a subject based on whether the subject is indicated as having a viral infection or bacterial infection. In some embodiments, this method may comprise receiving a report indicating whether the subject has a viral infection or a bacterial infection, wherein the report is based on the gene expression data obtained by measuring the amount of RNA transcripts encoded by JUP, SUCLG2, IFI27, FCER1A, HESX1, SMARCD3, ICAM1, and EBI3, and treating a subject based on whether the subject is indicated as having an viral infection or bacterial infection. In some embodiments the method may comprise: (a) identifying the patient as having increased JUP, SUCLG2, IFI27, and FCER1A, and HESX1 expression, and treating the subject with anti-viral therapy; or (b) identifying the patient as having increased SMARCD3, ICAM1, EBI3 expression, and treating the subject with an anti-bacterial therapy.

A subject indicated as having a viral infection may be treated by administering a therapeutically effective dose of an antiviral agent, such as a broad-spectrum antiviral agent, an antiviral vaccine, a neuraminidase inhibitor (e.g., zanamivir (Relenza) and oseltamivir (Tamiflu)), a nucleoside analogue (e.g., acyclovir, zidovudine (AZT), and lamivudine), an antisense antiviral agent (e.g., phosphorothioate antisense antiviral agents (e.g., Fomivirsen (Vitravene) for cytomegalovirus retinitis), morpholino antisense antiviral agents), an inhibitor of viral uncoating (e g, Amantadine and rimantadine for influenza, Pleconaril for rhinoviruses), an inhibitor of viral entry (e.g., Fuzeon for HIV), an inhibitor of viral assembly (e.g., Rifampicin), or an antiviral agent that stimulates the immune system (e.g., interferons). Exemplary antiviral agents include Abacavir, Aciclovir, Acyclovir, Adefovir, Amantadine, Amprenavir, Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

A subject indicated as having a bacterial infection may be treated by administering a therapeutically effective dose of an antibiotic. Antibiotics may include broad spectrum, bactericidal, or bacteriostatic antibiotics. Exemplary antibiotics include aminoglycosides such as Amikacin, Amikin, Gentamicin, Garamycin, Kanamycin, Kantrex, Neomycin, Neo-Fradin, Netilmicin, Netromycin, Tobramycin, Nebcin, Paromomycin, Humatin, Streptomycin, Spectinomycin(Bs), and Trobicin; ansamycins such as Geldanamycin, Herbimycin, Rifaximin, and Xifaxan; carbacephems such as Loracarbef and Lorabid; carbapenems such as Ertapenem, Invanz, Doripenem, Doribax, Imipenem/Cilastatin, Primaxin, Meropenem, and Merrem; cephalosporins such as Cefadroxil, Duricef, Cefazolin, Ancef, Cefalotin or Cefalothin, Keflin, Cefalexin, Keflex, Cefaclor, Distaclor, Cefamandole, Mandol, Cefoxitin, Mefoxin, Cefprozil, Cefzil, Cefuroxime, Ceftin, Zinnat, Cefixime, Cefdinir, Cefditoren, Cefoperazone, Cefotaxime, Cefpodoxime, Ceftazidime, Ceftibuten, Ceftizoxime, Ceftriaxone, Cefepime, Maxipime, Ceftaroline fosamil, Teflaro, Ceftobiprole, and Zeftera; glycopeptides such as Teicoplanin, Targocid, Vancomycin, Vancocin, Telavancin, Vibativ, Dalbavancin, Dalvance, Oritavancin, and Orbactiv; lincosamides such as Clindamycin, Cleocin, Lincomycin, and Lincocin; lipopeptides such as Daptomycin and Cubicin; macrolides such as Azithromycin, Zithromax, Sumamed, Xithrone, Clarithromycin, Biaxin, Dirithromycin, Dynabac, Erythromycin, Erythocin, Erythroped, Roxithromycin, Troleandomycin, Tao, Telithromycin, Ketek, Spiramycin, and Rovamycine; monobactams such as Aztreonam and Azactam; nitrofurans such as Furazolidone, Furoxone, Nitrofurantoin, Macrodantin, and Macrobid; oxazolidinones such as Linezolid, Zyvox, VRSA, Posizolid, Radezolid, and Torezolid; penicillins such as Penicillin V, Veetids (Pen-Vee-K), Piperacillin, Pipracil, Penicillin G, Pfizerpen, Temocillin, Negaban, Ticarcillin, and Ticar; penicillin combinations such as Amoxicillin/clavulanate, Augmentin, Ampicillin/sulbactam, Unasyn, Piperacillin/tazobactam, Zosyn, Ticarcillin/clavulanate, and Timentin; polypeptides such as Bacitracin, Colistin, Coly-Mycin-S, and Polymyxin B; quinolones/fluoroquinolones such as Ciprofloxacin, Cipro, Ciproxin, Ciprobay, Enoxacin, Penetrex, Gatifloxacin, Tequin, Gemifloxacin, Factive, Levofloxacin, Levaquin, Lomefloxacin, Maxaquin, Moxifloxacin, Avelox, Nalidixic acid, NegGram, Norfloxacin, Noroxin, Ofloxacin, Floxin, Ocuflox Trovafloxacin, Trovan, Grepafloxacin, Raxar, Sparfloxacin, Zagam, Temafloxacin, and Omniflox; sulfonamides such as Amoxicillin, Novamox, Amoxil, Ampicillin, Principen, Azlocillin, Carbenicillin, Geocillin, Cloxacillin, Tegopen, Dicloxacillin, Dynapen, Flucloxacillin, Floxapen, Mezlocillin, Mezlin, Methicillin, Staphcillin, Nafcillin, Unipen, Oxacillin, Prostaphlin, Penicillin G, Pentids, Mafenide, Sulfamylon, Sulfacetamide, Sulamyd, Bleph-10, Sulfadiazine, Micro-Sulfon, Silver sulfadiazine, Silvadene, Sulfadimethoxine Di-Methox, Albon, Sulfamethizole, Thiosulfil Forte, Sulfamethoxazole, Gantanol, Sulfanilimide, Sulfasalazine, Azulfidine, Sulfisoxazole, Gantrisin, Trimethoprim-Sulfamethoxazole (Co-trimoxazole) (TMP-SMX), Bactrim, Septra, Sulfonamidochrysoidine, and Prontosil; tetracyclines such as Demeclocycline, Declomycin, Doxycycline, Vibramycin, Minocycline, Minocin, Oxytetracycline, Terramycin, Tetracycline and Sumycin, Achromycin V, and Steclin; drugs against mycobacteria such as Clofazimine, Lamprene, Dapsone, Avlosulfon, Capreomycin, Capastat, Cycloserine, Seromycin, Ethambutol, Myambutol, Ethionamide, Trecator, Isoniazid, I.N.H., Pyrazinamide, Aldinamide, Rifampicin, Rifadin, Rimactane, Rifabutin, Mycobutin, Rifapentine, Priftin, and Streptomycin; others antibiotics such as Arsphenamine, Salvarsan, Chloramphenicol, Chloromycetin, Fosfomycin, Monurol, Monuril, Fusidic acid, Fucidin, Metronidazole, Flagyl, Mupirocin, Bactroban, Platensimycin, Quinupristin/Dalfopristin, Synercid, Thiamphenicol, Tigecycline, Tigacyl, Tinidazole, Tindamax Fasigyn, Trimethoprim, Proloprim, and Trimpex.

Methods for administering and dosages for administering the therapeutics listed above are known in the art or can be derived from the art.

Kits

Also provided by this disclosure are kits for practicing the subject methods, as described above. In some embodiments, the kit may contain reagents for measuring the amount of RNA transcripts encoded by JUP, SUCLG2, IFI27, FCER1A, HESX1, SMARCD3, ICAM1, and EBI3. In some embodiments, the kit may comprise, for each RNA transcript, a sequence-specific oligonucleotide that hybridizes to the transcript. In some embodiments, the sequence-specific oligonucleotide may be biotinylated and/or labeled with an optically-detectable moiety. In some embodiments, the kit may comprise, for each RNA transcript, a pair of PCR primers that amplify a sequence from the RNA transcript, or cDNA made from the same. In some embodiments, the kit may comprise an array of oligonucleotide probes, wherein the array comprises, for each RNA transcript, at least one sequence-specific oligonucleotide that hybridizes to the transcript. The oligonucleotide probes may be spatially addressable on the surface of a planar support, or tethered to optically addressable beads, for example.

In embodiments in which a quantitative isothermal amplification method is used, the kit may comprise reagents comprise multiple reaction vessels, each vessel comprising at least one (e.g., 2, 3, 4, 5, or 6) sequence-specific isothermal amplification primer that hybridizes to a single transcript, e.g., a transcript from a single gene selected from JUP, SUCLG2, IFI27, FCER1A, HESX1, SMARCD3, ICAM1, and EBI3, or cDNA made from the same. As such, in some embodiments, the kit may contain at least 8 reaction vessels, where each reaction vessels contain one or more primers for detection of an RNA transcript encoded by a single gene. In some embodiments, the kit may contain reagents for measuring the amount of up to a total of 30 or 50 RNA transcripts.

In some embodiments, the kit may contain reagents for measuring the amount of RNA transcripts of a set of any number of genes (e.g., at least 2, at least 3, at least 4, at least 5, at least 6, or at least 7 genes, up to 30 or 50 genes), where the set of genes includes any pair of genes listed in Table 2 as well as optionally other genes (e.g., at least 2, at least 3, at least 4, at least 5, at least 6, or at least 7 other genes) that independently are or are not listed on Table 1. For example, the kit may comprise, for each RNA transcript, a pair of PCR primers that amplify a sequence from the RNA transcript, or cDNA made from the same.

The various components of the kit may be present in separate containers or certain compatible components may be precombined into a single container, as desired.

In addition to the above-mentioned components, the subject kit may further include instructions for using the components of the kit to practice the subject method.

### Additional Embodiments

In any embodiment, the method can be practiced by measuring the amount of RNA transcripts encoded by than the eight listed genes, e.g., by measuring the amount of RNA transcripts encoded by 2, 3, 4, 5, 6, or 7 of the listed genes. The total number of transcripts measured in some embodiments may be 30 or 50 RNA in some embodiments.

In addition, other genes can be analyzed in addition to the eight listed genes or subset thereof. For example, in any embodiment, the method may further comprise measuring the amount of RNA transcripts of other genes listed in Table 1 below.

In some embodiments, the method may be practiced by measuring the amount of RNA transcripts of a set of any number of genes (e.g., at least 2, at least 3, at least 4, at least 5, at least 6, or at least 7 genes, up to 30 or 50 genes), where the set of genes includes any pair of genes listed in Table 2 as well as optionally other genes (e.g., at least 2, at least 3, at least 4, at least 5, at least 6, or at least 7 other genes) that are independently listed or not listed in Table 1.

In some embodiments, the method may further comprise measuring the amount of RNA transcripts encoded by CEACAM1, ZDHHC19, C9orf95, GNA15, BATF, C3AR1, KIAA1370, TGFBI, MTCH1, RPGRIP1, and HLA-DPB1 in addition to the listed genes. In these embodiments, increased expression of the CEACAM1, ZDHHC19, C9orf95, GNA15, BATF, and C3AR1 biomarkers and decreased expression of the KIAA1370, TGFBI, MTCH1, RPGRIP1, and HLA-DPB1 indicate that the subject has sepsis as described in WO2016145426. Thus, the present method can be used as an integrated decision model for the treatment of both bacterial and viral infections.

### Examples

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

Materials and Methods

Systematic Datasets Search

A systematic search was performed in NIH Gene Expression Omnibus (GEO) and European Bioinformatics Institute (EBI) ArrayExpress for public human microarray genome-wide expression studies of pneumonia or other respiratory infections. Datasets were excluded if they (i) were nonclinical, (ii) were performed using tissues other than whole blood or PBMCs, (iii) did not have at least 4 healthy samples, or (iv) did not have sufficient pathogen labels to identify whether the causal agent was bacterial or viral.

All microarray data were renormalized from raw data (when available) using standard methods. Affymetrix arrays were normalized using GC robust multiarray average (gcRMA) (on arrays with mismatch probes) or RMA. Illumina, Agilent, GE, and other commercial arrays were normalized via normal-exponential background correction followed by quantile normalization. Custom arrays were not renormalized and were used as is. Data were log₂-transformed, and a fixed-effect model was used to summarize probes to genes within each study. Within each study, cohorts assayed with different microarray types were treated as independent.

COCONUT Conormalization

Out of 43 datasets that matched inclusion criteria and profiled respiratory infections, only 12 of these datasets contained both bacterial and viral infections, and only a single one contained intracellular bacterial, extracellular bacterial, and viral infections. Because of the difference in background measurements for these different arrays (owing to the use of different platforms), it is difficult to conduct analyses between all 43 datasets without getting significantly skewed results due to the batch effects. In order to make use of these data, Combat CO-Normalization Using conTrols (COCONUT)², which allows for co-normalization of expression data without changing the distribution of genes between studies and without any bias towards sample diagnosis, was used. It applies a modified version of the ComBat empirical Bayes normalization method²⁶ that only assumes an equal distribution between control samples. Briefly, the healthy controls from each cohort undergo ComBat conormalization without covariates, and the ComBat estimated parameters are acquired for each dataset's healthy samples. These parameters are then applied to the diseased samples in each dataset, which causes all samples to assume the same background distribution while still retaining the relative distance between healthy and diseased samples in each dataset.

Calculation of Signature Score

A previously described signature score^{1,2,5,24,25} was used to perform disease classification. The signature score (Sᵢ) is calculated as the geometric mean of the genes that are positively correlated with the response variable (in this case, bacterial infections) minus the geometric mean of the negatively correlated genes (Eq. 1).

$${S}_{i} = {( {\prod }_{geneepos} {x}_{i} ( gene ) )}^{\frac{1}{\vert pos \vert}} - {( {\prod }_{geneeneg} {x}_{i} ( gene ) )}^{\frac{1}{\vert neg \vert}} \qquad ( 1 )$$

Abridged Best Subset Selection

This method combines a greedy backward search with an exhaustive search. Performing a greedy search alone would be computationally feasible, but because of the nature of the greedy algorithm it does not ensure that the best possible combination of genes for diagnostic purposes is found. On the other hand, because best subset selection is an exhaustive search, it will always select the optimal combination of genes; however, the computational cost of best subset selection increases exponentially, so running it on more than ˜20 genes was infeasible. The Abridged Best Subset Selection (Abridged BSS) is a way to combine the strengths of both of these methods.

First, a greedy backward search on the initial gene list was run. Briefly, the search involves taking the starting gene set and calculating the AUROC after individually removing each of the genes. The search further involves identifying which gene's removal leads to the largest increase in AUROC, and then permanently removing that gene from the set. This same strategy is then applied to the new gene set, once again removing the gene whose exclusion results in the largest increase in AUROC. In a typical greedy backward search, this step would be repeated until a point where removing any gene results in a reduction of AUROC that is greater than some pre-defined threshold is reached. However, in this case, the greedy backward search is simply run until enough genes are eliminated to be able to perform best subset selection (in this case, this cutoff was 20 genes).

The best subset selection can be run on the abridged gene list. Briefly, the diagnostic power of every possible combination of the genes is assessed by calculating the signature scores for each combination and reporting the corresponding AUROC. Next, for every unique number of total genes, the subset of genes that produces the best AUC is reported. This results in a list of the best signatures for each number of total genes, from which the final gene signature can be selected.

Derivation of the 8 Gene Signature Using MANATEE

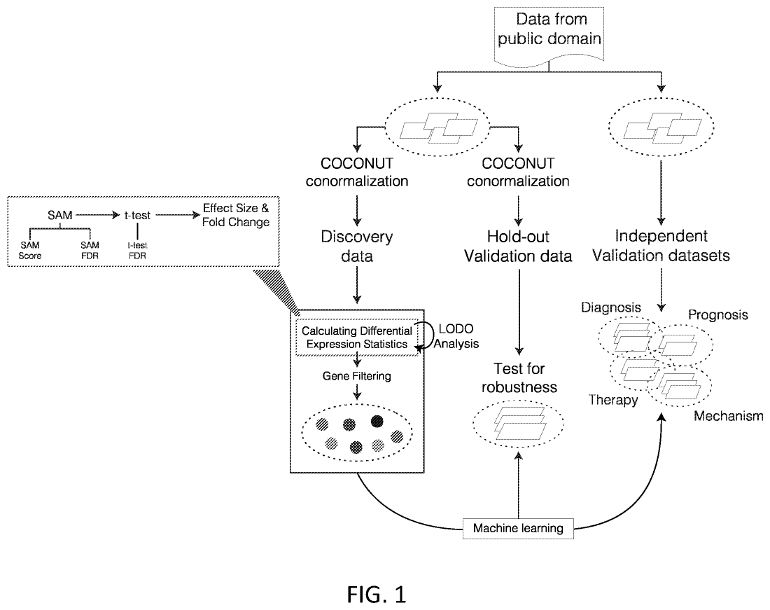

The Discovery respiratory infection cohorts were analyzed using Multicohort ANalysis of AggregaTed gEne Expression or MANATEE (FIG. 1). MANATEE was developed as a multicohort analysis framework to allow integration of a large number of independent heterogeneous datasets in a single gene expression analyses than was possible with the previous workflow. MANATEE starts by randomly splitting data into discovery and held-out validation. Here 70% of the data was assigned to discovery and the remaining 30% to held-out validation. Next, the discovery and held-out validation data are independently normalize using COCONUT. Within the discovery data, for each gene 5 measures of differential expression between cases and controls were calculated: (1) SAM score (from the Significance Analysis of Microarrays)²⁷, (2) corresponding SAM local FDR, (3) Benjamini-Hochberg FDR corrected P value (from running a t-test)²⁸, (4) effect size, and (5) fold change. The effect size was estimated as Hedges' adjusted g, which accounts for small sample bias. A leave-one-dataset-out (LODO) analysis was also performed, wherein each dataset that accounted for at least 5% of the samples was individually removed from the discovery data, and the differential expression statistics were re-calculated for each iteration of the discovery data with one dataset left out. In order for a gene to be selected by MANATEE, it must not only pass the set thresholds in the statistics calculated in the full discovery data, but it must also pass the thresholds for each iteration of the discovery data with one dataset removed. This prevents any single dataset from exerting too strong of a presence on the selection of genes.

Next, the top 100 genes with the highest SAM score were selected. In order to select only those genes that were highly diagnostic, an Abridged BSS (described above) was performed on these genes. From the results of the Abridged BSS, a 15-gene signature (the signature with the max AUROC) and an 8-gene signature (the smallest signature that was within the 95% CI of the max AUROC signature) were selected to test in Hold-out Validation. Both signatures had equivalent AUROCs, so the 8-gene signature was chosen for next steps.

### Results

The systematic search for gene expression microarray or RNA-seq cohorts that profiled patients with intracellular bacterial, extracellular bacterial, or viral infections resulting in febrile symptoms^{3,4} identified 43 whole blood (WB) cohorts and 9 peripheral blood mononuclear cell (PBMC) that met the inclusion criteria.⁵⁻²² The 43 independent WB cohorts were comprised of 1963 non-healthy patient samples (562 extracellular bacterial infections, 320 intracellular bacterial infections, and 1081 viral infections), whereas the 9 independent PBMC cohorts were comprised of 417 non-healthy patient samples (172 extracellular bacterial infections, 11 intracellular bacterial infections, and 234 viral infections). These data included both children and adults from a broad spectrum of geographic regions. 28 WB datasets consisting of 1419 infected samples (348 extracellular bacterial infections, 280 intracellular bacterial infections, and 791 viral infections) were used as discovery cohorts, and the remaining 15 WB datasets consisting of 544 non-healthy samples (214 extracellular bacterial infections, 40 intracellular bacterial infections, and 290 viral infections) were used as independent validation cohorts. Four datasets (3 WB and 1 PBMC) that had no healthy samples, but that had patients with bacterial or viral infections, which were used as independent validation cohorts, were identified.

Selecting Top Differentially Expressed Genes with MANATEE

In order to utilize all of the data that had been collected, a multicohort analysis framework called Multicohort ANalysis of AggregaTed gEne Expression (MANATEE) (FIG. 1) was developed. In this framework, 70% of the data was randomly assigned to the “discovery” cohort and the other 30% as “hold-out validation”. Next, COCONUT normalization was applied across all discovery cohorts.² COCONUT was applied separately to the discovery and held-out validation data. After co-normalization, there were 6086 common genes across all datasets. After calculating differential expression statistics for each gene, the framework involved filtering by selecting the top 100 genes (58 up in bacterial infection, 42 up in viral infection) with the highest SAM (Significance Analysis of Microarrays) score. Using the previously described signature score model^{1,2,5,24,25,} these 100 genes were used to classify samples as having bacterial or viral infections, resulting in an AUROC of 0.874 (95% CI 0.854 to 0.894) in Discovery data.

TABLE 1
(Genes in the final 8-gene signature are underlined)

| Genes up in bacterial | Genes up in viral |
|---|---|
| infection | infection |
| EHD1 | HERC6 |
| CPD | JUP |
| CD44 | IFIT1 |
| ZDHHC3 | LY6E |
| JAK3 | TMEM123 |
| SORT1 | MX1 |
| NDST2 | NUP205 |
| GAS7 | CAPN2 |
| GRB10 | TARBP1 |
| IRAK3 | IFI44L |
| SOCS3 | ICAM2 |
| PADI2 | OAS2 |
| ATP9A | SUCLG2 |
| UGCG | OAS1 |
| ACAA1 | RSAD2 |
| SMARCD3 | DNMT1 |
| CR1 | TLR7 |
| STAT5B | ST3GAL5 |
| IL4R | DRAP1 |
| MICAL1 | IFIT3 |
| ICAM1 | GNLY |
| PRKAR2A | PRF1 |
| BMX | GZMB |
| ALOX5AP | MX2 |
| CA4 | PTPRO |
| SOD2 | IFI44 |
| DACH1 | ISG20 |
| MAPK14 | IL2RB |
| WDFY3 | IFI27 |
| PADI4 | RABGAP1L |
| ZNF281 | RIN2 |
| VNN1 | LY86 |
| HDAC4 | BLVRA |
| NARF | CD86 |
| NFKBIA | ITGA4 |
| IL1R2 | FCER1A |
| PGD | IFIT2 |
| CDK5RAP2 | EPHB1 |
| CD82 | SAMD9 |
| FES | IFIT5 |
| MKNK1 | HESX1 |
| ALCAM | CCL8 |
| PHTF1 | |
| BCL6 | |
| SORL1 | |
| PROS1 | |
| FLOT2 | |
| LIMK2 | |
| DYSF | |
| ENTPD7 | |
| VSIG4 | |
| SMPDL3A | |
| DAAM2 | |
| FKBP5 | |
| EBI3 | |
| SLC1A3 | |
| MMP9 | |
| ALPL | |

Deriving the 8 Gene Signature with MANATEE

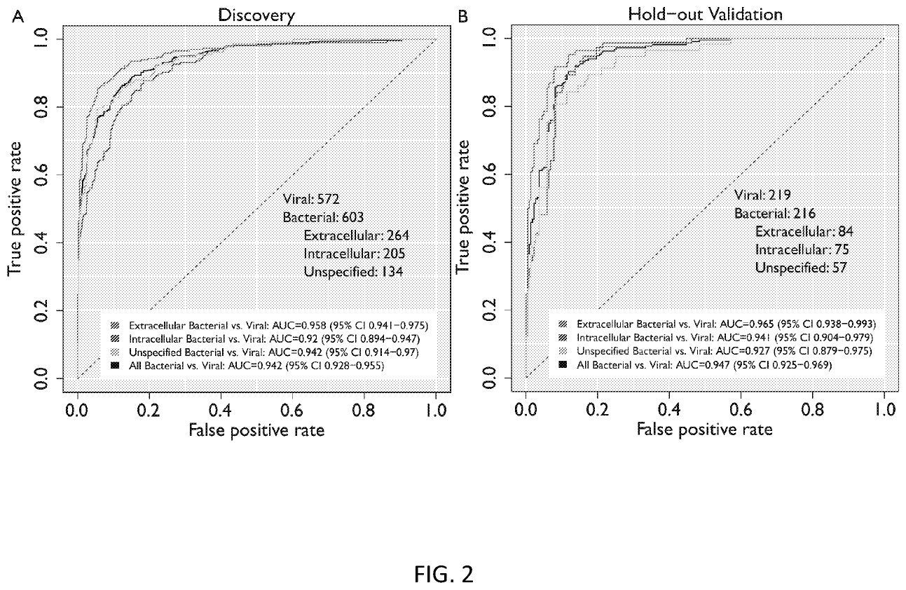

The next step involved running an Abridged Best Subset Selection (Abridged BSS) on the list of 100 genes, which consists of first running a greedy backward search to select the top 20 best genes, and then running an exhaustive search on those 20 genes. Running the Abridged BSS on the current gene list allowed identification of most important genes within the signature for distinguishing bacterial and viral infections. From the results of the Abridged BSS, two signatures were selected for testing: the signature that had the maximum AUROC in Discovery [15 genes, AUROC=0.951 (95% CI 0.939 to 0.964)] and the smallest signature that was within the 95% confidence interval of the max AUROC signature [8 genes, AUROC=0.942 (95% CI 0.928 to 0.955); FIG. 2A]. In held-out validation, the 15-gene signature had an AUROC of 0.948 (95% CI 0.926 to 0.969) and the 8-gene signature had an AUROC of 0.947 (95% CI 0.925 to 0.969) (FIG. 2B). Because both signatures had virtually equivalent AUROCs in held-out validation, the smaller 8-gene signature was chosen for further investigation. In this signature, there were 3 genes that were higher in bacterial infections (SMARCD3, ICAM1, EBI3) and 5 genes that were higher in viral infections (JUP, SUCLG2, IFI27, FCER1A, HESX1).

Validating in Independent in Silico Cohorts

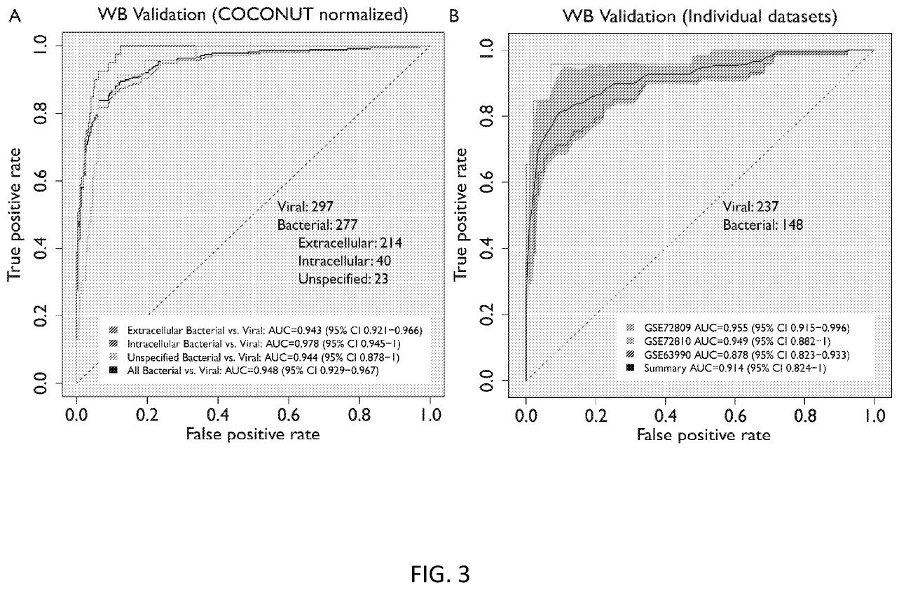

In order to verify that the results were broadly applicable and were not simply overfit to the training data, the performance of the 8-gene signature was tested in a series of completely independent cohorts. The 15 WB datasets were normalized with healthy samples that had been left out of discovery and held-out validation using COCONUT. These data included 544 non-healthy samples (214 extracellular bacterial infections, 40 intracellular bacterial infections, and 290 viral infections). The 8-signature had an AUROC of 0.948 (95% CI 0.929 to 0.967), 0.943 (95% CI 0.921 to 0.966), and 0.978 (95% CI 0.945 to 1) for distinguishing all bacterial vs. viral infections, extracellular bacterial vs. viral infections, and intracellular bacterial vs. viral infections, respectively (FIG. 3A). The 8-gene signature was further validated in 3 WB datasets that had both bacterial and viral infections but no healthy samples. In GSE72809 the AUROC was 0.955 (95% CI 0.915 to 0.996), in GSE72810 the AUROC was 0.949 (95% CI 0.882 to 1), in GSE63990 the AUROC was 0.878 (95% CI 0.823 to 0.933), and the summary AUC was 0.914 (95% CI 0.824 to 1) (FIG. 3B).

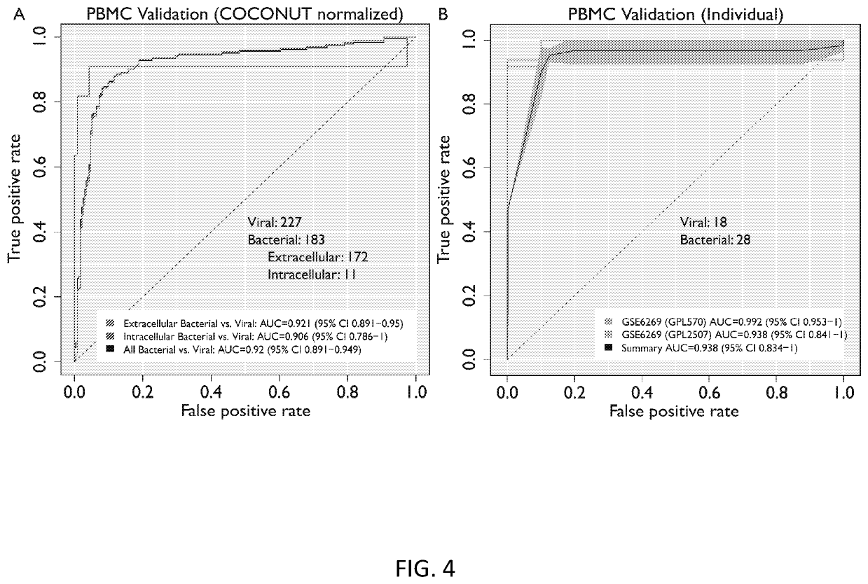

A similar validation was performed in the 9 PBMC cohorts, which included 417 non-healthy patient samples (172 extracellular bacterial infections, 11 intracellular bacterial infections, and 234 viral infections). After COCONUT normalization of these datasets, it was found that the signature had an AUROC of 0.92 (95% CI 0.891 to 0.949), 0.921 (95% CI 0.891 to 0.95), and 0.906 (95% CI 0.786 to 1) for distinguishing all bacterial vs. viral infections, extracellular bacterial vs. viral infections, and intracellular bacterial vs. viral infections, respectively (FIG. 4A). The 8-gene signature was further validated in a PBMC cohort with bacterial and viral infections but no healthy samples—this cohort was measured on two non-overlapping platforms (GPL570 and GPL2507). Therefore, a validation was done for each platform separately. In GSE6269GPL570 the AUROC was 0.992 (95% CI 0.953 to 1) and in GSE6269GPL2507 the AUROC was 0.938 (95% CI 0.841 to 1) (FIG. 4B).

Validating in Prospective Cohorts

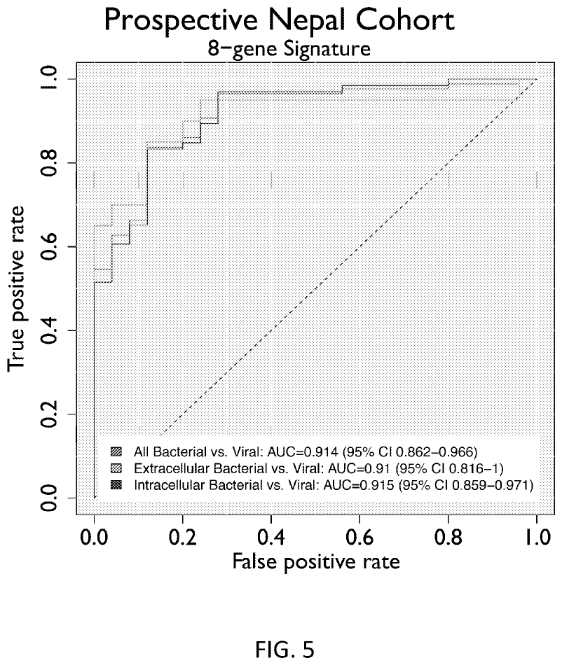

Finally, both the 7-gene and 8-gene signatures were profiled in a prospective cohort of 111 whole blood samples from Nepal using Fluidigm RT-PCR. It contains 25 viral infections, 15 extracellular bacterial infections, and 71 intracellular bacterial infections. Although 7-gene signature distinguished extracellular bacterial infections from viral infections with high accuracy (AUROC=0.886, 95% CI: 0.78-0.99), it had substantially lower accuracy in distinguishing intracellular bacterial infections from viral infections (AUROC=0.78, 95% CI: 0.68-0.88). The 7-gene signature had overall low accuracy in distinguishing bacterial and viral infections (AUROC=0.8, 95% CI: 0.72-0.89) (FIG. 5). In contrast, the 8-gene signature had an AUROC of 0.91 (95% CI 0.816 to 0.1) and 0.915 (95% CI 0.859 to 0.971) for distinguishing viral infections from extracellular and intracellular bacterial infections, respectively. Overall, the 8-gene signature had high accuracy in distinguishing bacterial and viral infection (AUROC=0.914, 95% CI 0.862 to 0.966). Together, these results give high confidence in the diagnostic power of this signature.

Two Gene Combinations

The Area Under the Receiver Operating Curve (AUROC) for each pairwise combination of genes listed in Table 1 was calculated. Table 2 below shows the AUROC for all pairwise combinations of genes that have an AUROC ≥0.80:

TABLE 2

| Gene 1 | Gene 2 | AUROC |
|---|---|---|
| ICAM1 | HERC6 | 0.89970891 |
| JAK3 | JUP | 0.88764511 |
| ICAM1 | JUP | 0.88737838 |
| GRB10 | JUP | 0.88658108 |
| JAK3 | HERC6 | 0.88606791 |
| JUP | SUCLG2 | 0.88290772 |
| EHD1 | JUP | 0.88217421 |
| SOCS3 | HERC6 | 0.88200315 |
| HERC6 | SUCLG2 | 0.87954169 |
| PADI2 | HERC6 | 0.87939092 |
| EBI3 | HERC6 | 0.87806596 |
| CD44 | JUP | 0.87697294 |
| SORT1 | HERC6 | 0.87620174 |
| CPD | JUP | 0.87618725 |
| ICAM1 | HESX1 | 0.87578135 |
| SOCS3 | HESX1 | 0.87498405 |
| SMARCD3 | HERC6 | 0.87482749 |
| SOD2 | HERC6 | 0.87463034 |
| EHD1 | HERC6 | 0.87398091 |
| NDST2 | JUP | 0.87397801 |
| SMARCD3 | JUP | 0.87306475 |
| NDST2 | HERC6 | 0.87303285 |
| ZDHHC3 | JUP | 0.87271973 |
| LIMK2 | HERC6 | 0.87264145 |
| ACAA1 | JUP | 0.87184706 |
| CPD | HERC6 | 0.87151944 |
| GRB10 | HERC6 | 0.87084681 |
| SOD2 | JUP | 0.87078013 |
| NFKBIA | HERC6 | 0.87073954 |
| EBI3 | JUP | 0.87064097 |
| PADI2 | JUP | 0.87015969 |
| LIMK2 | MX2 | 0.87008721 |
| MICAL1 | JUP | 0.86997704 |
| GAS7 | JUP | 0.86995674 |
| SORT1 | JUP | 0.86965812 |
| CPD | RIN2 | 0.86911596 |
| UGCG | HERC6 | 0.86802584 |
| JAK3 | HESX1 | 0.86795336 |
| SOCS3 | JUP | 0.86786058 |
| WDFY3 | HERC6 | 0.86758805 |
| JUP | CAPN2 | 0.86718795 |
| ATP9A | JUP | 0.86632397 |
| HERC6 | JUP | 0.86631818 |
| SORT1 | HESX1 | 0.86625729 |
| PRKAR2A | JUP | 0.86617611 |
| MICAL1 | HERC6 | 0.86564555 |
| WDFY3 | JUP | 0.865431 |
| ICAM1 | MX1 | 0.86525995 |
| NFKBIA | JUP | 0.8650454 |
| HERC6 | LY86 | 0.86472938 |
| CD44 | HERC6 | 0.86451194 |
| ACAA1 | HERC6 | 0.86372624 |
| IL4R | JUP | 0.86329715 |
| JUP | TARBP1 | 0.86325946 |
| SLC1A3 | HESX1 | 0.86296953 |
| SLC1A3 | HERC6 | 0.86293474 |
| ICAM1 | LY6E | 0.86258103 |
| STAT5B | JUP | 0.86245347 |
| JAK3 | TMEM123 | 0.86238678 |
| ICAM1 | TMEM123 | 0.86176634 |
| UGCG | JUP | 0.8615286 |
| BMX | HERC6 | 0.86136045 |
| ICAM1 | IFIT1 | 0.86129376 |
| ZDHHC3 | HERC6 | 0.86127927 |
| CPD | TMEM123 | 0.86126477 |
| PHTF1 | HERC6 | 0.86094585 |
| MAPK14 | HERC6 | 0.86060084 |
| GRB10 | HESX1 | 0.86046748 |
| GAS7 | HERC6 | 0.86043558 |
| LIMK2 | MX1 | 0.86043558 |
| SORT1 | RIN2 | 0.86043269 |
| HERC6 | ITGA4 | 0.86021814 |
| ATP9A | HERC6 | 0.86001519 |
| LIMK2 | IFIT1 | 0.8599717 |
| NDST2 | HESX1 | 0.85995431 |
| PRKAR2A | HERC6 | 0.8596006 |
| CPD | HESX1 | 0.85931357 |
| HERC6 | IL2RB | 0.85909613 |
| SORT1 | MX2 | 0.85907873 |
| PADI2 | HESX1 | 0.85905844 |
| SMARCD3 | HESX1 | 0.85888158 |
| STAT5B | HERC6 | 0.85884969 |
| LIMK2 | HESX1 | 0.85862065 |
| HERC6 | DNMT1 | 0.85855977 |
| HERC6 | TARBP1 | 0.85854817 |
| FES | JUP | 0.85798861 |
| UGCG | HESX1 | 0.85797412 |
| SOCS3 | MX2 | 0.85793353 |
| JUP | ITGA4 | 0.85787264 |
| EBI3 | HESX1 | 0.85779146 |
| ALOX5AP | HERC6 | 0.85759431 |
| JUP | DNMT1 | 0.85756532 |
| CPD | MX2 | 0.85744355 |
| JUP | NUP205 | 0.85735367 |
| CDK5RAP2 | HERC6 | 0.85715653 |
| MAPK14 | JUP | 0.85712173 |
| JAK3 | DRAP1 | 0.85707535 |
| ICAM1 | MX2 | 0.85704055 |
| HERC6 | CAPN2 | 0.85668105 |
| MKNK1 | HERC6 | 0.856481 |
| CD44 | TMEM123 | 0.85639402 |
| JUP | HESX1 | 0.85633024 |
| DACH1 | JUP | 0.85626645 |
| SOD2 | MX2 | 0.8560983 |
| NARF | JUP | 0.85591274 |
| JUP | ICAM2 | 0.85579967 |
| JUP | RABGAP1L | 0.85560832 |
| LIMK2 | JUP | 0.85548075 |
| HERC6 | CD86 | 0.85539378 |
| IRAK3 | HERC6 | 0.855301 |
| DAAM2 | JUP | 0.85476754 |
| SOCS3 | IFIT1 | 0.85472115 |
| JUP | TMEM123 | 0.85458199 |
| JUP | IL2RB | 0.85449501 |
| IL2RB | HESX1 | 0.85441383 |
| DAAM2 | HERC6 | 0.8541326 |
| IL4R | HERC6 | 0.85384267 |
| MKNK1 | JUP | 0.85381948 |
| CDK5RAP2 | JUP | 0.85324253 |
| IRAK3 | RIN2 | 0.85314395 |
| ZNF281 | JUP | 0.85314105 |
| BMX | HESX1 | 0.85303088 |
| SUCLG2 | HESX1 | 0.8529758 |
| EHD1 | CAPN2 | 0.8529729 |
| SMARCD3 | LY6E | 0.85284823 |
| WDFY3 | HESX1 | 0.85283373 |
| SORL1 | JUP | 0.85281054 |
| NFKBIA | HESX1 | 0.85275835 |
| FES | HERC6 | 0.85272936 |
| HDAC4 | HERC6 | 0.85264238 |
| HERC6 | NUP205 | 0.85263948 |
| EHD1 | LY6E | 0.85250902 |
| HERC6 | GNLY | 0.85248002 |
| SOD2 | IFIT1 | 0.85246843 |
| SORT1 | MX1 | 0.85229737 |
| SOCS3 | MX1 | 0.85226548 |
| PADI2 | MX2 | 0.8521843 |
| HERC6 | FCER1A | 0.85207703 |
| ALOX5AP | JUP | 0.85207123 |
| JUP | LY86 | 0.85193496 |
| ICAM1 | ISG20 | 0.85190307 |
| PROS1 | HERC6 | 0.85183059 |
| EHD1 | CPD | 0.8517784 |
| IRAK3 | JUP | 0.85174361 |
| SMPDL3A | HERC6 | 0.85169433 |
| IRAK3 | HESX1 | 0.85166533 |
| CR1 | JUP | 0.85152617 |
| EBI3 | LY6E | 0.85145079 |
| SORL1 | HERC6 | 0.85136091 |
| PHTF1 | HESX1 | 0.85118406 |
| ACAA1 | HESX1 | 0.85109418 |
| DNMT1 | HESX1 | 0.85105649 |
| BCL6 | JUP | 0.8510014 |
| MAPK14 | HESX1 | 0.85090863 |
| SLC1A3 | JUP | 0.85077236 |
| JAK3 | MX1 | 0.85074627 |
| SOD2 | HESX1 | 0.85065929 |
| CD82 | JUP | 0.85052882 |
| SOCS3 | RIN2 | 0.85038386 |
| SMPDL3A | HESX1 | 0.85037806 |
| BCL6 | HERC6 | 0.85037226 |
| EHD1 | HESX1 | 0.85037226 |
| DYSF | HERC6 | 0.85033167 |
| CPD | EPHB1 | 0.85000986 |
| JUP | PRF1 | 0.84994607 |
| LIMK2 | LY6E | 0.84974892 |
| SORT1 | LY6E | 0.84973443 |
| ICAM1 | DRAP1 | 0.84965325 |
| JAK3 | LY6E | 0.84960976 |
| JAK3 | IFIT1 | 0.84960106 |
| JUP | GNLY | 0.84949669 |
| JUP | GZMB | 0.84948509 |
| UGCG | IFIT1 | 0.84947929 |
| JAK3 | MX2 | 0.84934593 |
| SORT1 | IFIT1 | 0.84933723 |
| SOCS3 | LY6E | 0.84930244 |
| BMX | JUP | 0.84923576 |
| TARBP1 | HESX1 | 0.84922126 |
| HERC6 | ICAM2 | 0.84921836 |
| PADI2 | IFIT1 | 0.84917777 |
| ICAM1 | TLR7 | 0.84911109 |
| SMPDL3A | JUP | 0.84901541 |
| EHD1 | MX1 | 0.84891974 |
| ICAM1 | OAS2 | 0.84875158 |
| ZDHHC3 | HESX1 | 0.84865011 |
| JUP | PTPRO | 0.84858632 |
| FCER1A | HESX1 | 0.84847905 |
| GRB10 | TMEM123 | 0.84845586 |
| EHD1 | GRB10 | 0.84841817 |
| SOD2 | MX1 | 0.84838627 |
| SMARCD3 | BLVRA | 0.84834278 |
| NFKBIA | MX1 | 0.8482964 |
| EHD1 | DRAP1 | 0.848279 |
| SORT1 | BLVRA | 0.84808475 |
| CPD | IFIT1 | 0.84805286 |
| MICAL1 | HESX1 | 0.84804996 |
| GAS7 | HESX1 | 0.84801517 |
| DACH1 | HERC6 | 0.84800937 |
| VSIG4 | JUP | 0.84792529 |
| UGCG | MX1 | 0.8478876 |
| SOD2 | TMEM123 | 0.84746141 |
| NARF | HERC6 | 0.84741792 |
| WDFY3 | MX2 | 0.84741502 |
| NFKBIA | IFIT1 | 0.84738313 |
| CPD | RABGAP1L | 0.84727296 |
| GRB10 | RIN2 | 0.84723527 |
| NDST2 | TMEM123 | 0.84718018 |
| CR1 | HERC6 | 0.84715699 |
| HDAC4 | JUP | 0.84713669 |
| PGD | JUP | 0.84693085 |
| ICAM1 | BLVRA | 0.846725 |
| SMARCD3 | IFIT1 | 0.84664382 |
| SMARCD3 | MX1 | 0.84659453 |
| EHD1 | IFIT1 | 0.84641478 |
| ATP9A | HESX1 | 0.84629011 |
| ZNF281 | HERC6 | 0.84627851 |
| HERC6 | GZMB | 0.84599729 |
| PHTF1 | JUP | 0.8459538 |
| LY6E | SUCLG2 | 0.84589291 |
| EBI3 | IFIT1 | 0.84576245 |
| EHD1 | TMEM123 | 0.84574505 |
| SOD2 | LY6E | 0.84573055 |
| ICAM1 | NUP205 | 0.84571606 |
| TMEM123 | SUCLG2 | 0.84568997 |
| HERC6 | PTPRO | 0.84551891 |
| IFIT1 | SUCLG2 | 0.84548122 |
| SORT1 | DRAP1 | 0.84546092 |
| SMARCD3 | CAPN2 | 0.84539134 |
| EHD1 | CD44 | 0.84534205 |
| VSIG4 | HERC6 | 0.84533626 |
| HERC6 | PRF1 | 0.84530146 |
| PADI2 | MX1 | 0.8451739 |
| CD44 | HESX1 | 0.8451449 |
| SORT1 | TMEM123 | 0.84511011 |
| PADI2 | TMEM123 | 0.84507242 |
| PADI2 | LY6E | 0.84502024 |
| FLOT2 | JUP | 0.84489847 |
| SLC1A3 | IFIT1 | 0.84487237 |
| GNLY | HESX1 | 0.84485498 |
| MX1 | SUCLG2 | 0.84482599 |
| FLOT2 | HERC6 | 0.8447593 |
| NFKBIA | LY6E | 0.84459984 |
| EHD1 | TARBP1 | 0.8444027 |
| SOCS3 | TMEM123 | 0.84435921 |
| JUP | IFIT1 | 0.84434761 |
| UGCG | MX2 | 0.84432152 |
| ALOX5AP | HESX1 | 0.84431862 |
| NFKBIA | MX2 | 0.84420265 |
| SMPDL3A | RIN2 | 0.84417655 |
| ENTPD7 | JUP | 0.84411567 |
| BMX | IFIT1 | 0.84402579 |
| MKNK1 | HESX1 | 0.84401419 |
| PGD | HERC6 | 0.84355611 |
| DYSF | MX2 | 0.84353002 |
| PROS1 | JUP | 0.84331837 |
| IFI27 | FCER1A | 0.8431966 |
| SORL1 | MX2 | 0.84318501 |
| CPD | CAPN2 | 0.84314152 |
| JUP | ST3GAL5 | 0.84313572 |
| ALCAM | JUP | 0.84312122 |
| EBI3 | MX1 | 0.84307194 |
| CD82 | HERC6 | 0.84299656 |
| CPD | MX1 | 0.84297336 |
| JUP | CD86 | 0.84291248 |
| ICAM1 | PTPRO | 0.84288348 |
| GRB10 | DRAP1 | 0.84270663 |
| FES | HESX1 | 0.84270083 |
| IFIT1 | GNLY | 0.84258776 |
| EHD1 | NDST2 | 0.84257616 |
| LY86 | HESX1 | 0.84257616 |
| EHD1 | NUP205 | 0.84246019 |
| GRB10 | MX2 | 0.84230073 |
| ICAM1 | IFIT5 | 0.84219056 |
| IL4R | MX2 | 0.84213838 |
| PRKAR2A | TMEM123 | 0.84195862 |
| PROS1 | IFIT1 | 0.84193543 |
| EHD1 | MX2 | 0.84193253 |
| TMEM123 | LY86 | 0.84183395 |
| MAPK14 | IFIT1 | 0.84181656 |
| BCL6 | IFIT1 | 0.84177887 |
| MICAL1 | MX2 | 0.84172088 |
| GAS7 | RIN2 | 0.84170929 |
| BMX | LY6E | 0.84148314 |
| WDFY3 | TMEM123 | 0.84123091 |
| FES | MX2 | 0.84120771 |
| ICAM1 | SAMD9 | 0.84120771 |
| IFIT1 | LY86 | 0.84119032 |
| ICAM1 | RIN2 | 0.84108595 |
| BMX | MX1 | 0.84101636 |
| MX1 | LY86 | 0.84100187 |
| IRAK3 | IFIT1 | 0.84096998 |
| LY6E | FCER1A | 0.84073803 |
| DYSF | HESX1 | 0.84071774 |
| LIMK2 | OAS1 | 0.84068295 |
| CPD | EBI3 | 0.84064816 |
| CPD | LY6E | 0.8404974 |
| ICAM1 | OAS1 | 0.84034953 |
| ALOX5AP | IFIT1 | 0.84034084 |
| SMARCD3 | TMEM123 | 0.84027415 |
| JAK3 | NUP205 | 0.84026256 |
| IL2RB | RIN2 | 0.84023356 |
| SLC1A3 | RIN2 | 0.8400973 |
| UGCG | RIN2 | 0.83986246 |
| EHD1 | SUCLG2 | 0.83979868 |
| SMARCD3 | DRAP1 | 0.83963052 |
| WDFY3 | RIN2 | 0.83961892 |
| UGCG | TMEM123 | 0.83955224 |
| HERC6 | HESX1 | 0.83950875 |
| SOCS3 | SAMD9 | 0.83946526 |
| CPD | BLVRA | 0.83944207 |
| PRKAR2A | HESX1 | 0.83941887 |
| GAS7 | LY86 | 0.83936379 |
| JUP | LY6E | 0.8393261 |
| ICAM1 | CAPN2 | 0.83924492 |
| GRB10 | IFIT1 | 0.83917534 |
| DAAM2 | HESX1 | 0.83913475 |
| EHD1 | ICAM2 | 0.83912605 |
| ICAM1 | ICAM2 | 0.83910575 |
| PRF1 | HESX1 | 0.83895499 |
| BCL6 | MX2 | 0.83884482 |
| ZDHHC3 | TMEM123 | 0.83874625 |
| WDFY3 | IFIT1 | 0.83871145 |
| CD44 | IFIT1 | 0.83867376 |
| IL4R | IFIT1 | 0.83864767 |
| ENTPD7 | HERC6 | 0.83862158 |
| PHTF1 | IFIT1 | 0.83861578 |
| HERC6 | TMEM123 | 0.83858099 |
| EHD1 | LY86 | 0.83855489 |
| IFIT1 | TARBP1 | 0.8385462 |
| ICAM1 | TARBP1 | 0.83850851 |
| HDAC4 | HESX1 | 0.83845922 |
| UGCG | LY6E | 0.83841283 |
| IRAK3 | MX2 | 0.83836354 |
| SLC1A3 | MX1 | 0.83830266 |
| EHD1 | GAS7 | 0.83826497 |
| CPD | NDST2 | 0.83822438 |
| CDK5RAP2 | HESX1 | 0.8381519 |
| CPD | NUP205 | 0.838149 |
| ITGA4 | HESX1 | 0.83811711 |
| ALOX5AP | LY6E | 0.83791996 |
| JUP | MX1 | 0.83789096 |
| MAPK14 | TMEM123 | 0.83782428 |
| CPD | PTPRO | 0.83773151 |
| CPD | DRAP1 | 0.83771991 |
| MX1 | FCER1A | 0.83765612 |
| LY6E | LY86 | 0.83756915 |
| MKNK1 | RIN2 | 0.83756045 |
| CD44 | CAPN2 | 0.83754595 |
| IL4R | HESX1 | 0.83748507 |
| NUP205 | HESX1 | 0.83747057 |
| EHD1 | ZDHHC3 | 0.83743868 |
| NDST2 | CAPN2 | 0.83720384 |
| DYSF | IFIT1 | 0.83718934 |
| GRB10 | LY6E | 0.83716905 |
| GNLY | RIN2 | 0.83712556 |
| TMEM123 | CAPN2 | 0.83709077 |
| BCL6 | MX1 | 0.83700379 |
| EHD1 | JAK3 | 0.8369603 |
| CR1 | HESX1 | 0.83694001 |
| IFIT1 | IL2RB | 0.83682404 |
| SMARCD3 | MX2 | 0.83680664 |
| ENTPD7 | HESX1 | 0.83675736 |
| SORL1 | IFIT1 | 0.83675156 |
| ACAA1 | CAPN2 | 0.83674866 |
| SMARCD3 | LY86 | 0.83672836 |
| EHD1 | ACAA1 | 0.83668487 |
| SORT1 | SAMD9 | 0.83667908 |
| EHD1 | DNMT1 | 0.83663849 |
| MX1 | TARBP1 | 0.8365921 |
| ACAA1 | TMEM123 | 0.8365776 |
| SOCS3 | OAS1 | 0.8365718 |
| IFIT1 | GZMB | 0.83646163 |
| STAT5B | MX2 | 0.83644424 |
| NDST2 | NUP205 | 0.83644134 |
| HDAC4 | IFIT1 | 0.83638335 |
| IFIT1 | DNMT1 | 0.83625868 |
| GAS7 | TMEM123 | 0.83622389 |
| IFIT1 | FCER1A | 0.83615431 |
| ALOX5AP | MX1 | 0.83607603 |
| MAPK14 | LY6E | 0.83600935 |
| ICAM2 | HESX1 | 0.83596296 |
| LIMK2 | OAS2 | 0.83584119 |
| IRAK3 | MX1 | 0.83573392 |
| LY6E | TARBP1 | 0.83570782 |
| VSIG4 | HESX1 | 0.83568173 |
| IL4R | MX1 | 0.83566434 |
| SLC1A3 | LY6E | 0.83565564 |
| SMARCD3 | PTPRO | 0.83564984 |
| HERC6 | RABGAP1L | 0.83554837 |
| DYSF | JUP | 0.83553677 |
| EHD1 | EBI3 | 0.83553677 |
| IFIT1 | PRF1 | 0.83551937 |
| JUP | DRAP1 | 0.83538601 |
| CPD | JAK3 | 0.83536281 |
| NFKBIA | TMEM123 | 0.83536281 |
| FES | IFIT1 | 0.83534542 |
| NDST2 | IFIT1 | 0.83530773 |
| NDST2 | MX1 | 0.83521495 |
| CD44 | ICAM1 | 0.83521205 |
| NDST2 | DRAP1 | 0.83517146 |
| PADI4 | HERC6 | 0.83500041 |
| HERC6 | EPHB1 | 0.83499461 |
| CPD | ICAM1 | 0.83496272 |
| PHTF1 | MX1 | 0.83493952 |
| ALCAM | HERC6 | 0.83485544 |
| STAT5B | HESX1 | 0.83466409 |
| SUCLG2 | DRAP1 | 0.8346235 |
| CPD | LY86 | 0.83454812 |
| MAPK14 | MX1 | 0.83446694 |
| JUP | FCER1A | 0.83443215 |
| CPD | SUCLG2 | 0.83436257 |
| CR1 | IFIT1 | 0.8342466 |
| BMX | MX2 | 0.83420311 |
| LIMK2 | TMEM123 | 0.83413933 |
| FLOT2 | MX2 | 0.83411033 |
| CD44 | MX1 | 0.83406105 |
| JUP | BLVRA | 0.83405815 |
| SOCS3 | BLVRA | 0.83401466 |
| PROS1 | HESX1 | 0.83395667 |
| PHTF1 | LY6E | 0.83392768 |
| CD44 | LY6E | 0.83391898 |
| MKNK1 | MX2 | 0.83391898 |
| STAT5B | IFIT1 | 0.83391318 |
| SOCS3 | DRAP1 | 0.83390739 |
| MICAL1 | IFIT1 | 0.83388709 |
| ICAM1 | ST3GAL5 | 0.8338581 |
| DACH1 | HESX1 | 0.83381751 |
| ZDHHC3 | DRAP1 | 0.83378272 |
| CD44 | NUP205 | 0.83373053 |
| TARBP1 | RIN2 | 0.83357687 |
| EHD1 | PTPRO | 0.83350149 |
| ZNF281 | IFIT1 | 0.83340871 |
| MICAL1 | TMEM123 | 0.83340871 |
| DYSF | RIN2 | 0.83336523 |
| GAS7 | IFIT1 | 0.83335363 |
| GRB10 | MX1 | 0.83328695 |
| EBI3 | TMEM123 | 0.83325215 |
| GRB10 | ICAM1 | 0.83323476 |
| CDK5RAP2 | IFIT1 | 0.83321736 |
| FLOT2 | IFIT1 | 0.83319127 |
| ZDHHC3 | IFIT1 | 0.83317097 |
| JUP | TLR7 | 0.83311299 |
| MAPK14 | RIN2 | 0.8330724 |
| SMPDL3A | IFIT1 | 0.8330579 |
| CD44 | SUCLG2 | 0.83304341 |
| NDST2 | LY6E | 0.83298542 |
| CD44 | RIN2 | 0.83297093 |
| UGCG | OAS1 | 0.83295063 |
| FES | MX1 | 0.83292744 |
| BCL6 | LY6E | 0.83290424 |
| MKNK1 | IFIT1 | 0.83289265 |
| EHD1 | VSIG4 | 0.83288395 |
| IL1R2 | HERC6 | 0.83286365 |
| PGD | HESX1 | 0.83284046 |
| ICAM1 | CCL8 | 0.83283466 |
| CD86 | HESX1 | 0.83279407 |
| HERC6 | ST3GAL5 | 0.83275058 |
| ATP9A | IFIT1 | 0.83274478 |
| SORT1 | OAS1 | 0.83272739 |
| STAT5B | EBI3 | 0.83263461 |
| LIMK2 | SAMD9 | 0.83263171 |
| SMARCD3 | CD86 | 0.83262881 |
| PADI4 | JUP | 0.83259402 |
| HERC6 | RIN2 | 0.83256793 |
| PROS1 | MX1 | 0.83254474 |
| ICAM1 | EPHB1 | 0.83246935 |
| SUCLG2 | MX2 | 0.83242297 |
| DYSF | MX1 | 0.83238528 |
| ACAA1 | IFIT1 | 0.83236208 |
| BCL6 | HESX1 | 0.83214754 |
| NUP205 | LY86 | 0.83204896 |
| MICAL1 | MX1 | 0.83185761 |
| FES | LY6E | 0.83181412 |
| IFIT1 | NUP205 | 0.83178513 |
| WDFY3 | LY6E | 0.83174454 |
| TARBP1 | MX2 | 0.83171555 |
| JAK3 | RIN2 | 0.83165176 |
| IRAK3 | LY6E | 0.83164597 |
| FKBP5 | HERC6 | 0.83162857 |
| IFIT1 | ICAM2 | 0.83161697 |
| STAT5B | TMEM123 | 0.83153579 |
| DNMT1 | MX2 | 0.83148651 |
| NUP205 | CAPN2 | 0.83148361 |
| MICAL1 | LY6E | 0.83135024 |
| SOCS3 | OAS2 | 0.83122267 |
| IFIT1 | CD86 | 0.83121978 |
| LIMK2 | RIN2 | 0.83121688 |
| ATP9A | TMEM123 | 0.83119948 |
| CD44 | NDST2 | 0.83117919 |
| JUP | RIN2 | 0.83116759 |
| CD44 | MX2 | 0.83104292 |
| EHD1 | PRKAR2A | 0.83097044 |
| PADI2 | DRAP1 | 0.83083997 |
| SORL1 | TMEM123 | 0.83078489 |
| DYSF | LY6E | 0.83077329 |
| LY6E | IL2RB | 0.83075589 |
| LIMK2 | IFIT5 | 0.8307385 |
| PADI2 | SAMD9 | 0.81485927 |
| GAS7 | SUCLG2 | 0.81483898 |
| SORL1 | LY86 | 0.81483028 |
| IRAK3 | IFI44L | 0.81482738 |
| NFKBIA | DRAP1 | 0.81478679 |
| DNMT1 | LY86 | 0.81476939 |
| STAT5B | RIN2 | 0.81469111 |
| ICAM1 | DACH1 | 0.81463023 |
| CPD | PADI2 | 0.81460414 |
| STAT5B | TARBP1 | 0.81456934 |
| EHD1 | CR1 | 0.81452296 |
| RSAD2 | FCER1A | 0.81450266 |
| CD44 | CD86 | 0.81448527 |
| CD44 | GAS7 | 0.81440119 |
| EBI3 | RSAD2 | 0.8143403 |
| MICAL1 | LY86 | 0.81429971 |
| IL1R2 | LY6E | 0.81427942 |
| MX2 | ITGA4 | 0.81425043 |
| SUCLG2 | ISG20 | 0.81421853 |
| SOCS3 | EPHB1 | 0.81421564 |
| ZDHHC3 | NUP205 | 0.81420114 |
| DAAM2 | MX2 | 0.81416635 |
| NFKBIA | IFI44 | 0.81412866 |
| SORT1 | ICAM1 | 0.81411706 |
| MX1 | EPHB1 | 0.81407937 |
| SOD2 | IFIT2 | 0.81406777 |
| SOD2 | DRAP1 | 0.81400689 |
| RSAD2 | GNLY | 0.81399819 |
| FKBP5 | MX1 | 0.81399239 |
| CD82 | MX2 | 0.81398369 |
| TARBP1 | OAS2 | 0.8139605 |
| EHD1 | GNLY | 0.81392861 |
| GRB10 | SOD2 | 0.81390831 |
| WDFY3 | OAS2 | 0.81388512 |
| ENTPD7 | MX1 | 0.81387062 |
| ACAA1 | PTPRO | 0.81385613 |
| NDST2 | ACAA1 | 0.81384743 |
| SMARCD3 | ST3GAL5 | 0.81384453 |
| SUCLG2 | OAS1 | 0.81383003 |
| EHD1 | IL4R | 0.81382134 |
| PADI2 | EPHB1 | 0.81381844 |
| IRAK3 | OAS2 | 0.81373726 |
| JAK3 | SUCLG2 | 0.81369957 |
| CPD | RSAD2 | 0.81366478 |
| GRB10 | RABGAP1L | 0.81365898 |
| CD44 | TLR7 | 0.81364158 |
| IL2RB | IFI27 | 0.81362129 |
| IFIT1 | IFI27 | 0.81361839 |
| SOCS3 | RABGAP1L | 0.81360099 |
| FLOT2 | RIN2 | 0.8135807 |
| OAS1 | FCER1A | 0.813572 |
| GRB10 | OAS2 | 0.8135633 |
| SORT1 | IFIT2 | 0.81351112 |
| ALPL | IFIT1 | 0.81351112 |
| NDST2 | SOD2 | 0.81349662 |
| STAT5B | EPHB1 | 0.81348502 |
| LIMK2 | DRAP1 | 0.81347632 |
| NFKBIA | IFIT3 | 0.81345313 |
| CDK5RAP2 | MX2 | 0.81339515 |
| GRB10 | OAS1 | 0.81337195 |
| ALCAM | LY6E | 0.81326758 |
| ACAA1 | CD86 | 0.81323279 |
| BCL6 | OAS1 | 0.81322989 |
| PGD | TMEM123 | 0.81320379 |
| OAS1 | GNLY | 0.8131632 |
| CPD | GZMB | 0.81314001 |
| IFIT1 | DRAP1 | 0.81314001 |
| SLC1A3 | IFI44L | 0.81303854 |
| VNN1 | LY6E | 0.81296606 |
| CD44 | BLVRA | 0.81294286 |
| ICAM1 | PRF1 | 0.81291387 |
| ICAM2 | SUCLG2 | 0.81285588 |
| CAPN2 | DRAP1 | 0.81285298 |
| ZNF281 | EBI3 | 0.81285009 |
| CAPN2 | DNMT1 | 0.81282399 |
| TLR7 | HESX1 | 0.8127979 |
| ACAA1 | RIN2 | 0.8127892 |
| TMEM123 | PTPRO | 0.8127805 |
| PHTF1 | OAS2 | 0.8127776 |
| ICAM1 | ALCAM | 0.81276311 |
| SLC1A3 | BLVRA | 0.81267323 |
| MAPK14 | OAS2 | 0.81266453 |
| DACH1 | MX2 | 0.81265873 |
| SMPDL3A | MX2 | 0.81265004 |
| IL4R | OAS2 | 0.81260655 |
| SORT1 | EPHB1 | 0.81258915 |
| WDFY3 | OAS1 | 0.81258625 |
| ZDHHC3 | EBI3 | 0.81258625 |
| JAK3 | PROS1 | 0.81257466 |
| EHD1 | RABGAP1L | 0.81251377 |
| GRB10 | TARBP1 | 0.81250797 |
| NFKBIA | SAMD9 | 0.81245579 |
| SOD2 | RSAD2 | 0.81243839 |
| EHD1 | IRAK3 | 0.8124094 |
| NDST2 | ZNF281 | 0.812392 |
| SMARCD3 | ICAM2 | 0.8123862 |
| SORT1 | IFI27 | 0.81236591 |
| DNMT1 | DRAP1 | 0.81236011 |
| LIMK2 | CCL8 | 0.81233982 |
| CD44 | OAS1 | 0.81233112 |
| PROS1 | MX2 | 0.81229633 |
| EHD1 | NFKBIA | 0.81225864 |
| NDST2 | SAMD9 | 0.81224994 |
| VSIG4 | CAPN2 | 0.81222385 |
| CPD | ST3GAL5 | 0.81220355 |
| IFIT1 | LY6E | 0.81218905 |
| ENTPD7 | RIN2 | 0.81216586 |
| ATP9A | NUP205 | 0.81212237 |
| PADI2 | TARBP1 | 0.81210208 |
| JUP | SAMD9 | 0.81204699 |
| JAK3 | PADI2 | 0.8120296 |
| SOCS3 | CAPN2 | 0.8120267 |
| RSAD2 | IL2RB | 0.8120209 |
| EHD1 | SAMD9 | 0.81198611 |
| GRB10 | SUCLG2 | 0.81193682 |
| JAK3 | STAT5B | 0.81191363 |
| EHD1 | NARF | 0.81184114 |
| PRKAR2A | CAPN2 | 0.81183824 |
| TARBP1 | SUCLG2 | 0.81182375 |
| SMARCD3 | EPHB1 | 0.81176866 |
| EBI3 | ICAM2 | 0.81175996 |
| SUCLG2 | RIN2 | 0.81173097 |
| DNMT1 | SAMD9 | 0.81171068 |
| GAS7 | SMARCD3 | 0.81166139 |
| BMX | BLVRA | 0.81165269 |
| GNLY | SAMD9 | 0.8116411 |
| JAK3 | ICAM1 | 0.81159761 |
| HERC6 | MX2 | 0.81157151 |
| IL4R | OAS1 | 0.81154542 |
| SOD2 | LY86 | 0.81154542 |
| EBI3 | BLVRA | 0.81149033 |
| JAK3 | IFIT5 | 0.81148743 |
| UGCG | BLVRA | 0.81145554 |
| CPD | SORT1 | 0.81139176 |
| PADI2 | RABGAP1L | 0.81135987 |
| BMX | RSAD2 | 0.81135987 |
| PROS1 | RSAD2 | 0.81135987 |
| UGCG | IFIT2 | 0.81133377 |
| DYSF | OAS2 | 0.81131058 |
| CPD | PROS1 | 0.81128159 |
| CA4 | JUP | 0.8112468 |
| ENTPD7 | LY6E | 0.8112352 |
| FKBP5 | LY6E | 0.8112178 |
| EBI3 | IFI44 | 0.81121201 |
| JAK3 | EBI3 | 0.81119751 |
| SOD2 | TARBP1 | 0.81118301 |
| IRAK3 | ICAM1 | 0.81116272 |
| FES | OAS1 | 0.81116272 |
| TMEM123 | MX1 | 0.81113373 |
| SORT1 | CD86 | 0.81110473 |
| IL1R2 | MX1 | 0.81107284 |
| IL1R2 | MX2 | 0.81106414 |
| ICAM1 | RABGAP1L | 0.81102645 |
| GRB10 | ACAA1 | 0.81101196 |
| GAS7 | GRB10 | 0.81100326 |
| DYSF | RSAD2 | 0.81097717 |
| GZMB | LY86 | 0.81096557 |
| ZDHHC3 | SUCLG2 | 0.81093948 |
| JAK3 | MICAL1 | 0.81093948 |
| ICAM2 | MX2 | 0.81089599 |
| MAPK14 | SAMD9 | 0.81087279 |
| RIN2 | FCER1A | 0.8108467 |
| ATP9A | BLVRA | 0.810838 |
| ICAM1 | DAAM2 | 0.81081481 |
| BMX | IFIT5 | 0.81079161 |
| WDFY3 | BLVRA | 0.81078581 |
| PADI2 | LY86 | 0.81055967 |
| GZMB | PTPRO | 0.81052198 |
| EBI3 | TARBP1 | 0.81050749 |
| NDST2 | ISG20 | 0.81049009 |
| CA4 | HESX1 | 0.81048719 |
| GNLY | IFI44 | 0.8104466 |
| TARBP1 | RSAD2 | 0.8104379 |
| ACAA1 | SUCLG2 | 0.81040601 |
| BMX | ISG20 | 0.81037122 |
| VNN1 | MX1 | 0.81031903 |
| DYSF | TMEM123 | 0.81026105 |
| GRB10 | PADI2 | 0.81022626 |
| HDAC4 | OAS1 | 0.81018567 |
| BMX | IFI44L | 0.81010159 |
| OAS2 | GNLY | 0.81008419 |
| IRAK3 | CCL8 | 0.8100755 |
| BMX | IFI27 | 0.8100755 |
| ICAM1 | ZNF281 | 0.8100697 |
| CDK5RAP2 | OAS2 | 0.8100523 |
| DNMT1 | BLVRA | 0.81002621 |
| BCL6 | OAS2 | 0.81000302 |
| PRKAR2A | EBI3 | 0.80999142 |
| BMX | CCL8 | 0.80996532 |
| VSIG4 | LY86 | 0.80996532 |
| DACH1 | DRAP1 | 0.80994503 |
| JAK3 | SORT1 | 0.80994213 |
| EHD1 | RSAD2 | 0.80990734 |
| IRAK3 | CAPN2 | 0.80990444 |
| IL2RB | CCL8 | 0.80990154 |
| NDST2 | TLR7 | 0.80988415 |
| ZDHHC3 | NDST2 | 0.80985805 |
| ALOX5AP | OAS2 | 0.80984646 |
| EHD1 | HDAC4 | 0.80984646 |
| ALCAM | RIN2 | 0.80984356 |
| EBI3 | IFIT3 | 0.80981746 |
| ZDHHC3 | GAS7 | 0.80981746 |
| ISG20 | LY86 | 0.80980877 |
| IFI44L | LY86 | 0.80979137 |
| CPD | NARF | 0.80971309 |
| SORT1 | EBI3 | 0.80970149 |
| OAS1 | PRF1 | 0.80969859 |
| TLR7 | GNLY | 0.80965221 |
| PADI4 | EBI3 | 0.80964641 |
| UGCG | EBI3 | 0.80960292 |
| OAS2 | IL2RB | 0.80954783 |
| GRB10 | ISG20 | 0.80953913 |
| BMX | IFIT3 | 0.80953623 |
| SMARCD3 | SUCLG2 | 0.80953044 |
| GRB10 | ICAM2 | 0.80951304 |
| JUP | OAS1 | 0.80949854 |
| SMPDL3A | OAS1 | 0.80949854 |
| NDST2 | OAS1 | 0.80947535 |
| JAK3 | UGCG | 0.80945795 |
| NDST2 | LY86 | 0.80945216 |
| SOCS3 | PTPRO | 0.80937098 |
| ICAM1 | PADI4 | 0.80933039 |
| LIMK2 | EPHB1 | 0.80933039 |
| STAT5B | NUP205 | 0.80931879 |
| NFKBIA | CAPN2 | 0.80931299 |
| PRKAR2A | SUCLG2 | 0.8093014 |
| NUP205 | PTPRO | 0.8093014 |
| IRAK3 | DRAP1 | 0.8092927 |
| STAT5B | DRAP1 | 0.80926081 |
| GAS7 | SOD2 | 0.80923181 |
| NUP205 | RABGAP1L | 0.80919992 |
| CR1 | OAS1 | 0.80916803 |
| IL4R | EBI3 | 0.80914484 |
| LY6E | ST3GAL5 | 0.80911874 |
| PRKAR2A | RIN2 | 0.80907815 |
| LIMK2 | TLR7 | 0.80906656 |
| CD44 | ICAM2 | 0.80906076 |
| GAS7 | ACAA1 | 0.80905496 |
| MAPK14 | RSAD2 | 0.80904336 |
| NARF | EBI3 | 0.80903466 |
| ATP9A | EBI3 | 0.80901437 |
| SORL1 | NUP205 | 0.80897668 |
| CDK5RAP2 | OAS1 | 0.80894189 |
| CD44 | PROS1 | 0.80893609 |
| NDST2 | PTPRO | 0.80893319 |
| UGCG | IFI27 | 0.80891869 |
| ALOX5AP | DRAP1 | 0.8088839 |
| FES | OAS2 | 0.808881 |
| ZDHHC3 | PTPRO | 0.80879982 |
| GRB10 | STAT5B | 0.80878823 |
| NARF | TMEM123 | 0.80871864 |
| EBI3 | MX2 | 0.80868675 |
| PROS1 | OAS1 | 0.80865776 |
| CR1 | OAS2 | 0.80857948 |
| TARBP1 | PTPRO | 0.80856788 |
| MICAL1 | ICAM2 | 0.80855629 |
| ICAM1 | GNLY | 0.80852729 |
| LY6E | TLR7 | 0.8084867 |
| NFKBIA | IFIT5 | 0.8084838 |
| EHD1 | IL2RB | 0.80846641 |
| GNLY | IFI27 | 0.80844901 |
| BMX | TLR7 | 0.80839393 |
| IFI44L | GNLY | 0.80833014 |
| WDFY3 | CAPN2 | 0.80831855 |
| SOCS3 | TLR7 | 0.80828666 |
| HERC6 | OAS2 | 0.80826056 |
| EHD1 | PHTF1 | 0.80825766 |
| FES | LY86 | 0.80825766 |
| GRB10 | RSAD2 | 0.80825186 |
| SORT1 | SMARCD3 | 0.80824027 |
| JAK3 | PRKAR2A | 0.80823157 |
| CD44 | ATP9A | 0.80823157 |
| JUP | RSAD2 | 0.80818228 |
| WDFY3 | CCL8 | 0.80815909 |
| SOD2 | RABGAP1L | 0.80815329 |
| DRAP1 | IL2RB | 0.8081301 |
| IL2RB | BLVRA | 0.8081185 |
| VSIG4 | PTPRO | 0.8081011 |
| DRAP1 | LY86 | 0.80804022 |
| FLOT2 | TMEM123 | 0.80798803 |
| SOD2 | IFI44L | 0.80794744 |
| MX1 | ST3GAL5 | 0.80793295 |
| MICAL1 | PTPRO | 0.80791555 |
| CPD | IFIT3 | 0.80789815 |
| FES | DRAP1 | 0.80788946 |
| GAS7 | PADI2 | 0.80785177 |
| UGCG | CAPN2 | 0.80784017 |
| JUP | IFI44L | 0.80784017 |
| CD44 | SAMD9 | 0.80783437 |
| SOD2 | NUP205 | 0.80782857 |
| SMARCD3 | SAMD9 | 0.80780538 |
| IRAK3 | IFIT3 | 0.80777639 |
| OAS2 | HESX1 | 0.80776189 |
| IRAK3 | LY86 | 0.80775609 |
| PADI2 | IFIT5 | 0.8077358 |
| IFI44 | FCER1A | 0.80773 |
| ALOX5AP | BLVRA | 0.8077097 |
| IL1R2 | RSAD2 | 0.80769231 |
| ZDHHC3 | SOD2 | 0.80765752 |
| EHD1 | IFIT3 | 0.80763142 |
| ZDHHC3 | ICAM2 | 0.80757344 |
| NUP205 | ICAM2 | 0.80756764 |
| SORT1 | ICAM2 | 0.80752415 |
| CR1 | CAPN2 | 0.80752125 |
| EBI3 | ST3GAL5 | 0.80751835 |
| CPD | IFIT2 | 0.80749806 |
| TLR7 | GZMB | 0.80748356 |
| IFI44 | IL2RB | 0.80746617 |
| ICAM1 | BMX | 0.80744297 |
| NUP205 | TARBP1 | 0.80738789 |
| TARBP1 | SAMD9 | 0.80737629 |
| MICAL1 | BLVRA | 0.80735889 |
| JAK3 | RABGAP1L | 0.8073473 |
| NDST2 | PADI2 | 0.8073241 |
| SORT1 | TARBP1 | 0.8073241 |
| EBI3 | SAMD9 | 0.8073212 |
| GRB10 | VSIG4 | 0.80730961 |
| EHD1 | ITGA4 | 0.80729511 |
| EHD1 | IFI44L | 0.80727192 |
| RSAD2 | LY86 | 0.80724292 |
| EBI3 | CD86 | 0.80720523 |
| DACH1 | EBI3 | 0.80720233 |
| UGCG | EPHB1 | 0.80715305 |
| HDAC4 | OAS2 | 0.80711246 |
| SOCS3 | IFI27 | 0.80710956 |
| MX1 | TLR7 | 0.80710086 |
| PROS1 | IFI27 | 0.80709216 |
| CD44 | ITGA4 | 0.80708636 |
| SORT1 | SUCLG2 | 0.80704577 |
| IFI44L | IL2RB | 0.80703708 |
| SUCLG2 | RSAD2 | 0.80703418 |
| MAPK14 | EPHB1 | 0.8069588 |
| PADI4 | RIN2 | 0.8069472 |
| BMX | IFI44 | 0.80691821 |
| TMEM123 | FCER1A | 0.80691241 |
| SMARCD3 | PRKAR2A | 0.80689501 |
| NUP205 | RIN2 | 0.80688342 |
| GNLY | CCL8 | 0.80688342 |
| JAK3 | VSIG4 | 0.80687182 |
| ACAA1 | TLR7 | 0.80683703 |
| GRB10 | MICAL1 | 0.80679644 |
| PADI2 | SUCLG2 | 0.80678194 |
| IL4R | CAPN2 | 0.80677034 |
| SUCLG2 | EPHB1 | 0.80675295 |
| PROS1 | OAS2 | 0.80672396 |
| SOD2 | IFI44 | 0.80666597 |
| NFKBIA | EPHB1 | 0.80663988 |
| SMPDL3A | DRAP1 | 0.80663118 |
| UGCG | TLR7 | 0.80661378 |
| STAT5B | PTPRO | 0.80661088 |
| NFKBIA | IFIT2 | 0.80660219 |
| ACAA1 | ICAM1 | 0.80658769 |
| GRB10 | CD86 | 0.80658769 |
| PADI4 | MX2 | 0.80657609 |
| UGCG | SMARCD3 | 0.8065674 |
| BMX | EBI3 | 0.8065645 |
| SOCS3 | NUP205 | 0.8065616 |
| NDST2 | DNMT1 | 0.8065558 |
| OAS2 | GZMB | 0.8065471 |
| PADI2 | CD86 | 0.80652971 |
| CPD | IFI44 | 0.80652101 |
| CA4 | MX1 | 0.80652101 |
| TARBP1 | CD86 | 0.80647752 |
| SMARCD3 | SORL1 | 0.80641663 |
| ALOX5AP | RSAD2 | 0.80639054 |
| IRAK3 | EBI3 | 0.80637025 |
| EHD1 | CCL8 | 0.80635575 |
| CPD | DNMT1 | 0.80631226 |
| SUCLG2 | CD86 | 0.80630356 |
| OAS1 | GZMB | 0.80630066 |
| EHD1 | CDK5RAP2 | 0.80626297 |
| VSIG4 | BLVRA | 0.80623978 |
| NDST2 | GZMB | 0.80622238 |
| ATP9A | SMARCD3 | 0.80619629 |
| SOD2 | PTPRO | 0.8061789 |
| ALOX5AP | SAMD9 | 0.8061731 |
| JAK3 | IFI44L | 0.8061528 |
| JAK3 | DNMT1 | 0.80613831 |
| UGCG | NUP205 | 0.80610352 |
| CA4 | LY6E | 0.80609482 |
| IFIT1 | ISG20 | 0.80608612 |
| DAAM2 | IFIT1 | 0.83070951 |
| TMEM123 | ICAM2 | 0.83067761 |
| JAK3 | BLVRA | 0.83064862 |
| PADI2 | RIN2 | 0.83063992 |
| WDFY3 | MX1 | 0.83061093 |
| SOCS3 | CCL8 | 0.83046597 |
| NDST2 | SMARCD3 | 0.83046307 |
| JAK3 | CAPN2 | 0.83046017 |
| IFIT1 | ITGA4 | 0.83043118 |
| MX1 | GNLY | 0.83040508 |
| EHD1 | CD86 | 0.83036449 |
| IL1R2 | IFIT1 | 0.8303239 |
| CDK5RAP2 | LY6E | 0.83023983 |
| JUP | ISG20 | 0.83023983 |
| ALCAM | HESX1 | 0.83010356 |
| PRKAR2A | IFIT1 | 0.83008327 |
| MAPK14 | MX2 | 0.83005137 |
| EHD1 | EPHB1 | 0.8299615 |
| MX1 | IL2RB | 0.82982813 |
| PROS1 | LY6E | 0.82981654 |
| CAPN2 | HESX1 | 0.82978754 |
| UGCG | OAS2 | 0.82974985 |
| CPD | ACAA1 | 0.82967737 |
| GZMB | HESX1 | 0.82966867 |
| UGCG | SAMD9 | 0.82965128 |
| ALOX5AP | MX2 | 0.82963678 |
| IFIT1 | HESX1 | 0.82949472 |
| CR1 | TMEM123 | 0.82932946 |
| CPD | SAMD9 | 0.82929177 |
| SOCS3 | RSAD2 | 0.82928887 |
| HERC6 | IFIT1 | 0.82926568 |
| TMEM123 | HESX1 | 0.82921349 |
| ACAA1 | BLVRA | 0.82910622 |
| SMARCD3 | TLR7 | 0.82908592 |
| ACAA1 | LY86 | 0.82903084 |
| IL1R2 | HESX1 | 0.82900764 |
| DACH1 | IFIT1 | 0.82899894 |
| IL1R2 | JUP | 0.82899025 |
| LIMK2 | IFIT2 | 0.82899025 |
| CR1 | RIN2 | 0.82895256 |
| EHD1 | ATP9A | 0.82894386 |
| PGD | IFIT1 | 0.82888008 |
| SORL1 | RIN2 | 0.82887428 |
| SORT1 | OAS2 | 0.82886848 |
| IL4R | LY6E | 0.82883659 |
| PHTF1 | TMEM123 | 0.8287815 |
| ZNF281 | HESX1 | 0.82865103 |
| DNMT1 | RIN2 | 0.82854376 |
| SORL1 | EBI3 | 0.82849447 |
| SOD2 | EPHB1 | 0.82845678 |
| HDAC4 | LY6E | 0.82843359 |
| EBI3 | OAS2 | 0.82833501 |
| FLOT2 | HESX1 | 0.82830602 |
| ACAA1 | MX2 | 0.82830022 |
| CPD | TARBP1 | 0.82828573 |
| EHD1 | BLVRA | 0.82822484 |
| HERC6 | BLVRA | 0.82822194 |
| NARF | HESX1 | 0.82820745 |
| CPD | SMARCD3 | 0.82819005 |
| SORL1 | HESX1 | 0.82815236 |
| BMX | TMEM123 | 0.82815236 |
| HDAC4 | MX1 | 0.82810597 |
| ICAM1 | CD86 | 0.82807408 |
| PADI2 | BLVRA | 0.82802479 |
| IFIT1 | RIN2 | 0.82791462 |
| SORL1 | MX1 | 0.82790013 |
| SOD2 | OAS1 | 0.82786823 |
| JUP | EPHB1 | 0.82777836 |
| LY6E | DNMT1 | 0.82773777 |
| GAS7 | DRAP1 | 0.82768848 |
| IRAK3 | TMEM123 | 0.82768558 |
| HDAC4 | RIN2 | 0.82764499 |
| CD44 | DRAP1 | 0.82763629 |
| LY6E | ITGA4 | 0.82763629 |
| GRB10 | CAPN2 | 0.8276102 |
| ZDHHC3 | LY6E | 0.82758701 |
| SOD2 | RIN2 | 0.82753482 |
| SORT1 | CCL8 | 0.82750583 |
| HDAC4 | TMEM123 | 0.82747104 |
| GRB10 | BLVRA | 0.82744494 |
| CD82 | IFIT1 | 0.82742465 |
| CPD | GRB10 | 0.82740725 |
| IFIT1 | CAPN2 | 0.82737536 |
| NFKBIA | OAS2 | 0.82736666 |
| IL4R | TMEM123 | 0.82729708 |
| MX1 | DNMT1 | 0.82726519 |
| ACAA1 | LY6E | 0.82716661 |
| CR1 | MX2 | 0.82715212 |
| CDK5RAP2 | MX1 | 0.82709703 |
| GAS7 | MX2 | 0.82707384 |
| CAPN2 | TARBP1 | 0.82705644 |
| MX1 | ITGA4 | 0.82704774 |
| NARF | IFIT1 | 0.82694627 |
| CR1 | LY6E | 0.82694337 |
| CD82 | HESX1 | 0.82692308 |
| FLOT2 | MX1 | 0.82687379 |
| LIMK2 | IFIT3 | 0.82687379 |
| CPD | CD44 | 0.82685349 |
| TMEM123 | TARBP1 | 0.8268506 |
| PGD | MX2 | 0.82679261 |
| FES | RIN2 | 0.82675202 |
| HERC6 | TLR7 | 0.82671433 |
| VSIG4 | IFIT1 | 0.82669114 |
| EHD1 | ISG20 | 0.82667374 |
| ZDHHC3 | CAPN2 | 0.82663895 |
| FKBP5 | IFIT1 | 0.82662735 |
| MX2 | IL2RB | 0.82662155 |
| JAK3 | CCL8 | 0.82660996 |
| NDST2 | TARBP1 | 0.82656357 |
| EHD1 | SORT1 | 0.82653458 |
| NFKBIA | OAS1 | 0.82647079 |
| CR1 | MX1 | 0.82637222 |
| MX1 | CD86 | 0.82636062 |
| ALOX5AP | RIN2 | 0.82635192 |
| LY6E | GNLY | 0.82634902 |
| ZDHHC3 | MX1 | 0.82630843 |
| EHD1 | ICAM1 | 0.82626784 |
| NDST2 | MX2 | 0.82624465 |
| SMARCD3 | OAS1 | 0.82622436 |
| JAK3 | OAS2 | 0.82614898 |
| TARBP1 | DRAP1 | 0.82610839 |
| JUP | MX2 | 0.82610549 |
| JAK3 | NDST2 | 0.8260417 |
| FLOT2 | LY6E | 0.82600981 |
| PRKAR2A | LY6E | 0.82598372 |
| ALOX5AP | TMEM123 | 0.82594313 |
| TARBP1 | LY86 | 0.82592863 |
| SMPDL3A | LY6E | 0.82592573 |
| GRB10 | NUP205 | 0.82583875 |
| SMARCD3 | RIN2 | 0.82582716 |
| CPD | TLR7 | 0.82578947 |
| JAK3 | TLR7 | 0.82577497 |
| HERC6 | LY6E | 0.82572858 |
| SOD2 | BLVRA | 0.82571989 |
| BCL6 | TMEM123 | 0.82568509 |
| PHTF1 | RIN2 | 0.8256793 |
| LY6E | HESX1 | 0.8256503 |
| SOCS3 | IFIT5 | 0.82563871 |
| IFIT1 | PTPRO | 0.82561551 |
| SORT1 | CAPN2 | 0.82561261 |
| ICAM1 | GZMB | 0.82559522 |
| SOD2 | OAS2 | 0.82558942 |
| GAS7 | LY6E | 0.82558652 |
| MX1 | ICAM2 | 0.82556333 |
| DACH1 | TMEM123 | 0.82552274 |
| UGCG | RSAD2 | 0.82543576 |
| SOCS3 | IFI44 | 0.82542706 |
| MX1 | PRF1 | 0.82538067 |
| PADI2 | CAPN2 | 0.82535168 |
| JAK3 | PTPRO | 0.82531399 |
| CD44 | SMARCD3 | 0.8252676 |
| ATP9A | MX1 | 0.8252676 |
| EHD1 | ST3GAL5 | 0.82516033 |
| GZMB | RIN2 | 0.82509655 |
| STAT5B | MX1 | 0.82500087 |
| ZDHHC3 | ICAM1 | 0.82499217 |
| MKNK1 | TMEM123 | 0.82497768 |
| GAS7 | CAPN2 | 0.82493709 |
| GRB10 | LY86 | 0.82493709 |
| SORT1 | RSAD2 | 0.82490519 |
| ATP9A | LY6E | 0.82486171 |
| EHD1 | UGCG | 0.82485591 |
| PRF1 | RIN2 | 0.82472834 |
| STAT5B | LY6E | 0.82472544 |
| PTPRO | HESX1 | 0.82469935 |
| NDST2 | RIN2 | 0.82465296 |
| LY6E | ICAM2 | 0.82463266 |
| GAS7 | BLVRA | 0.82462397 |
| JAK3 | ISG20 | 0.82437463 |
| SMPDL3A | MX1 | 0.82430795 |
| LY6E | RIN2 | 0.82425866 |
| MX1 | GZMB | 0.82424416 |
| LIMK2 | RSAD2 | 0.82417458 |
| SORL1 | LY6E | 0.82416588 |
| CPD | CD86 | 0.82416298 |
| TMEM123 | NUP205 | 0.82415429 |
| CD44 | PTPRO | 0.82415139 |
| PADI4 | IFIT1 | 0.8241195 |
| ICAM1 | LY86 | 0.8241108 |
| JAK3 | SAMD9 | 0.8241021 |
| UGCG | IFI44 | 0.82406441 |
| PGD | LY6E | 0.82403252 |
| MICAL1 | DRAP1 | 0.82400932 |
| CPD | ZDHHC3 | 0.82398033 |
| ZNF281 | TMEM123 | 0.82396294 |
| ST3GAL5 | HESX1 | 0.82389915 |
| JAK3 | TARBP1 | 0.82384117 |
| UGCG | CCL8 | 0.82380638 |
| EHD1 | STAT5B | 0.82378898 |
| CD44 | GRB10 | 0.82378318 |
| MX1 | HESX1 | 0.823731 |
| GAS7 | MX1 | 0.8237252 |
| CD82 | LY6E | 0.8237194 |
| ENTPD7 | IFIT1 | 0.82364982 |
| EHD1 | GZMB | 0.82363242 |
| BMX | OAS1 | 0.82362952 |
| NDST2 | SUCLG2 | 0.82360923 |
| SORT1 | LY86 | 0.82356284 |
| VNN1 | HERC6 | 0.82355414 |
| ZNF281 | MX1 | 0.82351645 |
| ACAA1 | MX1 | 0.82345557 |
| ZDHHC3 | MX2 | 0.82335989 |
| CPD | CCL8 | 0.8233106 |
| LY6E | NUP205 | 0.82329321 |
| SMPDL3A | TMEM123 | 0.82329321 |
| EHD1 | SMARCD3 | 0.82321783 |
| SOD2 | ISG20 | 0.82320333 |
| CD44 | PADI2 | 0.82315694 |
| PGD | MX1 | 0.82313665 |
| ICAM1 | RSAD2 | 0.82307866 |
| EHD1 | OAS2 | 0.82300328 |
| DAAM2 | TMEM123 | 0.82300038 |
| BMX | SAMD9 | 0.8229279 |
| HERC6 | MX1 | 0.82290181 |
| TARBP1 | BLVRA | 0.82289021 |
| VSIG4 | LY6E | 0.82288151 |
| LY6E | CD86 | 0.82282353 |
| HERC6 | DRAP1 | 0.82272785 |
| SORT1 | ISG20 | 0.82271626 |
| ATP9A | RIN2 | 0.82270756 |
| CPD | OAS1 | 0.82268726 |
| LY6E | CAPN2 | 0.82268146 |
| IRAK3 | BLVRA | 0.82256839 |
| TMEM123 | DNMT1 | 0.8225539 |
| PHTF1 | MX2 | 0.82247562 |
| LY6E | PRF1 | 0.82246692 |
| MX1 | NUP205 | 0.82245532 |
| SLC1A3 | MX2 | 0.82245242 |
| EHD1 | TLR7 | 0.82243793 |
| MKNK1 | MX1 | 0.82242343 |
| EBI3 | DRAP1 | 0.82235675 |
| SORL1 | EPHB1 | 0.82229006 |
| SMARCD3 | TARBP1 | 0.82228717 |
| GNLY | MX2 | 0.82226977 |
| CD44 | JAK3 | 0.82225237 |
| MKNK1 | LY6E | 0.82223788 |
| LIMK2 | IFI44 | 0.82217989 |
| ZDHHC3 | LY86 | 0.8221683 |
| PADI4 | HESX1 | 0.8221683 |
| PRKAR2A | MX1 | 0.8221596 |
| MICAL1 | CAPN2 | 0.82210451 |
| SORT1 | IFIT5 | 0.82209002 |
| GRB10 | EBI3 | 0.82207262 |
| FKBP5 | HESX1 | 0.82206972 |
| UGCG | IFI44L | 0.82206392 |
| GZMB | MX2 | 0.82197984 |
| MICAL1 | TARBP1 | 0.82197695 |
| ICAM1 | PRKAR2A | 0.82193056 |
| DAAM2 | LY6E | 0.82185228 |
| CPD | VSIG4 | 0.82178849 |
| CPD | GAS7 | 0.82178559 |
| ATP9A | ICAM1 | 0.8217653 |
| FES | TMEM123 | 0.8217624 |
| JAK3 | CD86 | 0.8217595 |
| EHD1 | RIN2 | 0.82169572 |
| NDST2 | GRB10 | 0.82166672 |
| MX1 | RIN2 | 0.82163193 |
| GAS7 | NUP205 | 0.82159714 |
| FKBP5 | JUP | 0.82158555 |
| ICAM1 | VSIG4 | 0.82157105 |
| BMX | RIN2 | 0.82156815 |
| JAK3 | OAS1 | 0.82151596 |
| IFIT1 | EPHB1 | 0.82141159 |
| LY6E | GZMB | 0.82140869 |
| PADI2 | OAS2 | 0.82126373 |
| IRAK3 | OAS1 | 0.82123474 |
| PADI2 | OAS1 | 0.82121444 |
| ZNF281 | LY6E | 0.82111297 |
| IFIT1 | RABGAP1L | 0.82111007 |
| LIMK2 | ISG20 | 0.82110717 |
| IL4R | RIN2 | 0.82104338 |
| ICAM1 | IFI44L | 0.82104338 |
| EHD1 | PADI2 | 0.82103759 |
| TMEM123 | GZMB | 0.82093901 |
| SOCS3 | IFIT3 | 0.82092162 |
| ZDHHC3 | RIN2 | 0.82089842 |
| CPD | MICAL1 | 0.82086363 |
| CD44 | LY86 | 0.82080275 |
| JAK3 | ICAM2 | 0.82079405 |
| EBI3 | NUP205 | 0.82072737 |
| SOD2 | SAMD9 | 0.82071577 |
| PRKAR2A | DRAP1 | 0.82070127 |
| LIMK2 | IFI44L | 0.82068968 |
| CD44 | TARBP1 | 0.82068388 |
| CPD | OAS2 | 0.82066648 |
| EHD1 | MICAL1 | 0.82065488 |
| JAK3 | LY86 | 0.8206085 |
| DACH1 | RIN2 | 0.82055631 |
| MX1 | CAPN2 | 0.82050992 |
| IL1R2 | RIN2 | 0.82044034 |
| EHD1 | DAAM2 | 0.82042874 |
| JAK3 | ST3GAL5 | 0.82042584 |
| EHD1 | OAS1 | 0.82040845 |
| GRB10 | SMARCD3 | 0.82034466 |
| SOCS3 | IFI44L | 0.82030407 |
| CR1 | ICAM1 | 0.82029828 |
| SORT1 | TLR7 | 0.82028958 |
| ZNF281 | MX2 | 0.82026638 |
| EHD1 | ALCAM | 0.82024899 |
| ICAM1 | IFIT2 | 0.8202084 |
| ICAM1 | IFIT3 | 0.8201997 |
| MAPK14 | OAS1 | 0.82016491 |
| ICAM1 | IFI44 | 0.82015041 |
| HDAC4 | MX2 | 0.82013302 |
| EBI3 | OAS1 | 0.82012432 |
| CD82 | MX1 | 0.82009533 |
| WDFY3 | DRAP1 | 0.82008953 |
| MAPK14 | DRAP1 | 0.82008953 |
| PROS1 | TMEM123 | 0.82003444 |
| PROS1 | RIN2 | 0.82002285 |
| EHD1 | SORL1 | 0.81997066 |
| BMX | DRAP1 | 0.81996776 |
| MX1 | PTPRO | 0.81992717 |
| LY6E | PTPRO | 0.81992137 |
| SMARCD3 | OAS2 | 0.81985759 |
| NDST2 | SORL1 | 0.81985179 |
| CPD | ICAM2 | 0.8198141 |
| ICAM1 | SORL1 | 0.8198112 |
| TMEM123 | GNLY | 0.8198083 |
| CDK5RAP2 | TMEM123 | 0.81976191 |
| SLC1A3 | RSAD2 | 0.81974452 |
| EPHB1 | HESX1 | 0.81973292 |
| CAPN2 | ICAM2 | 0.81971263 |
| FES | BLVRA | 0.81970393 |
| NFKBIA | RIN2 | 0.81960825 |
| BCL6 | RIN2 | 0.81957926 |
| SORT1 | IFI44 | 0.81953577 |
| EBI3 | PTPRO | 0.81953287 |
| DNMT1 | RABGAP1L | 0.81952707 |
| IFIT1 | ST3GAL5 | 0.81947779 |
| CD44 | MICAL1 | 0.8194198 |
| JUP | OAS2 | 0.81940241 |
| PADI2 | PTPRO | 0.81937921 |
| SMPDL3A | BLVRA | 0.81937051 |
| MICAL1 | NUP205 | 0.81936761 |
| CD44 | ZDHHC3 | 0.81934152 |
| GAS7 | EBI3 | 0.81932992 |
| PROS1 | RABGAP1L | 0.81932123 |
| IFIT1 | TLR7 | 0.81930963 |
| NDST2 | OAS2 | 0.81927774 |
| CPD | IFIT5 | 0.81923425 |
| CD44 | OAS2 | 0.81922265 |
| UGCG | IFIT5 | 0.81919366 |
| SLC1A3 | OAS1 | 0.81910668 |
| SMARCD3 | NUP205 | 0.8190168 |
| DAAM2 | MX1 | 0.81897332 |
| SOD2 | IFIT5 | 0.81896172 |
| GRB10 | CCL8 | 0.81895882 |
| IFI44L | FCER1A | 0.81893562 |
| EBI3 | GZMB | 0.81893562 |
| IL2RB | SAMD9 | 0.81889214 |
| PGD | RIN2 | 0.81886894 |
| IFIT1 | TMEM123 | 0.81883995 |
| NDST2 | ICAM2 | 0.81877037 |
| EBI3 | RIN2 | 0.81870079 |
| TMEM123 | CD86 | 0.8186341 |
| CD44 | VSIG4 | 0.81861961 |
| BMX | OAS2 | 0.81859351 |
| SORT1 | IFI44L | 0.81858192 |
| JAK3 | ACAA1 | 0.81857902 |
| VNN1 | IFIT1 | 0.81848044 |
| OAS2 | FCER1A | 0.81847754 |
| IRAK3 | RSAD2 | 0.81845145 |
| EHD1 | ZNF281 | 0.81837317 |
| TARBP1 | RABGAP1L | 0.81836447 |
| EBI3 | TLR7 | 0.81834998 |
| LY6E | RABGAP1L | 0.81832388 |
| NARF | MX1 | 0.81830359 |
| ICAM1 | SUCLG2 | 0.81829199 |
| SORT1 | PTPRO | 0.81819921 |
| EBI3 | ISG20 | 0.81818472 |
| OAS2 | LY86 | 0.81816732 |
| GRB10 | SAMD9 | 0.81816442 |
| NDST2 | ICAM1 | 0.81807455 |
| ACAA1 | DRAP1 | 0.81807455 |
| CA4 | IFIT1 | 0.81795278 |
| CPD | PRKAR2A | 0.81794118 |
| ACAA1 | ICAM2 | 0.81792958 |
| ZDHHC3 | JAK3 | 0.81792668 |
| OAS2 | SUCLG2 | 0.81791219 |
| SOD2 | CAPN2 | 0.8178745 |
| SOD2 | TLR7 | 0.8178658 |
| ATP9A | MX2 | 0.8178542 |
| STAT5B | CAPN2 | 0.81779332 |
| NDST2 | BLVRA | 0.81778752 |
| SOCS3 | ISG20 | 0.81773823 |
| NDST2 | STAT5B | 0.81773243 |
| EBI3 | CAPN2 | 0.81770344 |
| DACH1 | MX1 | 0.81769474 |
| ZDHHC3 | BLVRA | 0.81764546 |
| VNN1 | JUP | 0.81755268 |
| PRF1 | MX2 | 0.81755268 |
| VSIG4 | MX1 | 0.81752949 |
| IRAK3 | IFI27 | 0.81750049 |
| DACH1 | LY6E | 0.8174744 |
| ATP9A | DRAP1 | 0.81741641 |
| GAS7 | ICAM1 | 0.81741062 |
| IRAK3 | IFI44 | 0.81731784 |
| EHD1 | PROS1 | 0.81730624 |
| CD44 | EBI3 | 0.81730624 |
| VNN1 | HESX1 | 0.81730044 |
| UGCG | DRAP1 | 0.81729465 |
| PADI2 | EBI3 | 0.81728305 |
| CPD | ATP9A | 0.81726275 |
| SMARCD3 | ISG20 | 0.81720477 |
| SLC1A3 | IFI44 | 0.81715258 |
| RABGAP1L | HESX1 | 0.81714099 |
| PADI2 | ISG20 | 0.81713229 |
| GRB10 | PTPRO | 0.81712359 |
| ALCAM | IFIT1 | 0.81711779 |
| DAAM2 | RIN2 | 0.8171004 |
| SLC1A3 | OAS2 | 0.8170975 |
| LY6E | EPHB1 | 0.817083 |
| GAS7 | TARBP1 | 0.81702212 |
| EHD1 | WDFY3 | 0.81701342 |
| SOCS3 | IFIT2 | 0.81699022 |
| UGCG | ISG20 | 0.81692064 |
| GAS7 | PTPRO | 0.81685686 |
| GAS7 | CD86 | 0.81684526 |
| TMEM123 | PRF1 | 0.81681627 |
| EHD1 | SOCS3 | 0.81674379 |
| ALCAM | TMEM123 | 0.8166945 |
| GRB10 | EPHB1 | 0.8166945 |
| EHD1 | SOD2 | 0.81667131 |
| TMEM123 | IL2RB | 0.81667131 |
| NDST2 | GAS7 | 0.81663362 |
| UGCG | RABGAP1L | 0.81657563 |
| VNN1 | IFI27 | 0.81656693 |
| OAS1 | IL2RB | 0.81654084 |
| SLC1A3 | SAMD9 | 0.81650605 |
| MICAL1 | RIN2 | 0.81649735 |
| GAS7 | ICAM2 | 0.81646256 |
| PADI2 | NUP205 | 0.81645676 |
| PRKAR2A | MX2 | 0.81643647 |
| CAPN2 | SUCLG2 | 0.81643647 |
| PADI4 | LY6E | 0.81639588 |
| JAK3 | GRB10 | 0.81636109 |
| SORT1 | NUP205 | 0.81632919 |
| SORT1 | IFIT3 | 0.8163089 |
| NARF | MX2 | 0.8162973 |
| IRAK3 | SAMD9 | 0.8162973 |
| ZDHHC3 | SMARCD3 | 0.8162857 |
| CPD | ISG20 | 0.81626541 |
| PADI4 | MX1 | 0.81626541 |
| NARF | LY6E | 0.81625091 |
| NFKBIA | RSAD2 | 0.81625091 |
| JAK3 | EPHB1 | 0.81619003 |
| PHTF1 | SAMD9 | 0.81615814 |
| VSIG4 | TMEM123 | 0.81614654 |
| CR1 | EBI3 | 0.81611175 |
| UGCG | IFIT3 | 0.81605376 |
| SORL1 | RABGAP1L | 0.81605376 |
| IFIT1 | BLVRA | 0.81602767 |
| UGCG | ICAM1 | 0.81601317 |
| JAK3 | SMARCD3 | 0.81601027 |
| ACAA1 | TARBP1 | 0.81600448 |
| NFKBIA | IFI44L | 0.81597548 |
| STAT5B | ICAM1 | 0.81596389 |
| ICAM1 | DNMT1 | 0.8159291 |
| WDFY3 | SAMD9 | 0.81587111 |
| SORT1 | RABGAP1L | 0.81586531 |
| DYSF | OAS1 | 0.81586241 |
| VSIG4 | RIN2 | 0.81586241 |
| EHD1 | DACH1 | 0.81585951 |
| HERC6 | ISG20 | 0.81585661 |
| EBI3 | IFI44L | 0.81583052 |
| PRKAR2A | NUP205 | 0.81574934 |
| CD44 | SORT1 | 0.81572325 |
| EHD1 | PRF1 | 0.81571745 |
| CPD | ITGA4 | 0.81566816 |
| NFKBIA | ISG20 | 0.81564787 |
| JAK3 | GAS7 | 0.81564207 |
| ZNF281 | RIN2 | 0.81562177 |
| ACAA1 | NUP205 | 0.81558698 |
| CD44 | ACAA1 | 0.81558698 |
| LIMK2 | BLVRA | 0.81556959 |
| SOD2 | VSIG4 | 0.815529 |
| GAS7 | EPHB1 | 0.8155116 |
| LY6E | TMEM123 | 0.81549421 |
| MX1 | RABGAP1L | 0.81544782 |
| NUP205 | MX2 | 0.81542752 |
| CA4 | HERC6 | 0.81536664 |
| PHTF1 | OAS1 | 0.81535214 |
| TARBP1 | OAS1 | 0.81533475 |
| ATP9A | CAPN2 | 0.81532605 |
| PADI2 | TLR7 | 0.81523907 |
| MAPK14 | BLVRA | 0.81518109 |
| ICAM1 | PROS1 | 0.81516659 |
| NUP205 | SUCLG2 | 0.8151231 |
| PADI2 | CCL8 | 0.8151144 |
| CD82 | TMEM123 | 0.81505352 |
| SLC1A3 | TMEM123 | 0.81496944 |
| ALOX5AP | OAS1 | 0.81495784 |
| STAT5B | LY86 | 0.81493465 |
| JAK3 | ATP9A | 0.81489986 |
| SUCLG2 | SAMD9 | 0.81489696 |
| SOD2 | IFIT3 | 0.81488246 |
| TMEM123 | ST3GAL5 | 0.80412912 |
| SMARCD3 | IFIT5 | 0.80412042 |
| SLC1A3 | CCL8 | 0.80411173 |
| IFIT3 | GNLY | 0.80408273 |
| ATP9A | ICAM2 | 0.80407114 |
| VSIG4 | MX2 | 0.80404214 |
| MICAL1 | EBI3 | 0.80402765 |
| CR1 | RSAD2 | 0.80401025 |
| JUP | CCL8 | 0.80401025 |
| OAS2 | ITGA4 | 0.80399865 |
| NDST2 | PROS1 | 0.80395807 |
| PADI2 | IFIT3 | 0.80392617 |
| IL4R | IFI44L | 0.80391458 |
| TMEM123 | RABGAP1L | 0.80389718 |
| SMPDL3A | RSAD2 | 0.80385949 |
| MKNK1 | DRAP1 | 0.80384499 |
| WDFY3 | NUP205 | 0.8038392 |
| IL4R | IFI44 | 0.8038276 |
| HDAC4 | RSAD2 | 0.80379571 |
| GRB10 | IFI44L | 0.80378991 |
| JAK3 | IFIT3 | 0.80377831 |
| CAPN2 | PTPRO | 0.80376671 |
| BCL6 | IFIT3 | 0.80374932 |
| JAK3 | CR1 | 0.80372323 |
| SORL1 | OAS2 | 0.80371163 |
| PRKAR2A | OAS2 | 0.80368554 |
| GRB10 | SORL1 | 0.80365364 |
| PGD | BLVRA | 0.80365364 |
| TARBP1 | ISG20 | 0.80363915 |
| GNLY | PTPRO | 0.80362755 |
| MAPK14 | IFIT3 | 0.80358406 |
| SORL1 | TARBP1 | 0.80356956 |
| JUP | IFIT5 | 0.80356377 |
| ICAM2 | RIN2 | 0.80356377 |
| ACAA1 | EBI3 | 0.80355217 |
| SMARCD3 | IFIT3 | 0.80352898 |
| IFIT1 | CCL8 | 0.80352608 |
| BCL6 | RSAD2 | 0.80349418 |
| PADI2 | ST3GAL5 | 0.80349128 |
| FKBP5 | MX2 | 0.80348549 |
| JAK3 | IL4R | 0.80347389 |
| NDST2 | EBI3 | 0.80347389 |
| IFI44L | GZMB | 0.80346519 |
| ICAM1 | NARF | 0.80345359 |
| NDST2 | PRKAR2A | 0.8034391 |
| PHTF1 | IFIT5 | 0.80340141 |
| PADI2 | ICAM2 | 0.80338401 |
| CPD | PRF1 | 0.80336082 |
| CPD | UGCG | 0.80333472 |
| ACAA1 | OAS2 | 0.80332603 |
| ATP9A | OAS2 | 0.80328254 |
| TLR7 | IL2RB | 0.80324775 |
| UGCG | SUCLG2 | 0.80324195 |
| MICAL1 | TLR7 | 0.80323325 |
| CPD | DACH1 | 0.80321296 |
| GRB10 | IFI44 | 0.80318106 |
| CD44 | EPHB1 | 0.80318106 |
| ATP9A | OAS1 | 0.80317527 |
| DAAM2 | IFI27 | 0.80317237 |
| CPD | GNLY | 0.80317237 |
| EHD1 | IFIT5 | 0.80316947 |
| PADI4 | TMEM123 | 0.80316947 |
| WDFY3 | EPHB1 | 0.80316077 |
| CPD | FCER1A | 0.80315787 |
| MX1 | BLVRA | 0.80313178 |
| NARF | CAPN2 | 0.80310278 |
| CA4 | RIN2 | 0.80309989 |
| SORT1 | PADI2 | 0.80308829 |
| IL4R | RSAD2 | 0.8030535 |
| CPD | ALCAM | 0.80299261 |
| CAPN2 | LY86 | 0.80298681 |
| ATP9A | SAMD9 | 0.80297812 |
| SMPDL3A | IFI44L | 0.80297232 |
| EBI3 | PRF1 | 0.80292593 |
| IRAK3 | IFIT5 | 0.80292013 |
| FCER1A | CCL8 | 0.80291433 |
| NDST2 | CR1 | 0.80290274 |
| MAPK14 | TLR7 | 0.80289404 |
| IFI44 | LY86 | 0.80288244 |
| NDST2 | MICAL1 | 0.80286215 |
| JUP | IFI44 | 0.80285925 |
| DACH1 | SUCLG2 | 0.80285055 |
| SMPDL3A | OAS2 | 0.80283895 |
| SLC1A3 | IFIT3 | 0.80282736 |
| FES | CAPN2 | 0.80278097 |
| NDST2 | UGCG | 0.80276937 |
| SUCLG2 | LY86 | 0.80275777 |
| PADI2 | ATP9A | 0.80273168 |
| GRB10 | PRKAR2A | 0.80269399 |
| SLC1A3 | IFIT5 | 0.80269109 |
| TLR7 | PRF1 | 0.80269109 |
| GAS7 | MICAL1 | 0.80267949 |
| PRF1 | IFI44 | 0.8026331 |
| UGCG | GZMB | 0.80262441 |
| JAK3 | GZMB | 0.80258962 |
| IFIT1 | MX2 | 0.80256352 |
| PRF1 | SAMD9 | 0.80255772 |
| DYSF | IFI44 | 0.80255482 |
| BCL6 | IFI44 | 0.80253453 |
| CPD | IL4R | 0.80250844 |
| SMARCD3 | IFI44 | 0.80246495 |
| CD44 | ISG20 | 0.80246495 |
| MAPK14 | EBI3 | 0.80245625 |
| ISG20 | FCER1A | 0.80244465 |
| GAS7 | OAS2 | 0.80244175 |
| DYSF | CCL8 | 0.80243596 |
| ZDHHC3 | MICAL1 | 0.80240986 |
| IL4R | NUP205 | 0.80238667 |
| HDAC4 | IFI44L | 0.80238087 |
| PROS1 | EBI3 | 0.80233158 |
| PROS1 | TLR7 | 0.80230549 |
| ALOX5AP | ISG20 | 0.80229389 |
| PRF1 | LY86 | 0.8022765 |
| CR1 | IFI44L | 0.8022562 |
| HDAC4 | TLR7 | 0.8022475 |
| OAS1 | LY86 | 0.80223881 |
| CD44 | IFI44L | 0.80221271 |
| BMX | IFIT2 | 0.80220401 |
| CD44 | ALOX5AP | 0.80218952 |
| ZDHHC3 | TARBP1 | 0.80218372 |
| ALOX5AP | EPHB1 | 0.80215183 |
| SOD2 | CCL8 | 0.80214893 |
| IRAK3 | ISG20 | 0.80208804 |
| PRF1 | IFI27 | 0.80208515 |
| CPD | SOCS3 | 0.80207645 |
| SORL1 | FCER1A | 0.80207355 |
| CDK5RAP2 | IFI44L | 0.80204456 |
| HDAC4 | BLVRA | 0.80203586 |
| JUP | IFIT3 | 0.80202716 |
| CD44 | RSAD2 | 0.80201556 |
| MAPK14 | PTPRO | 0.80200976 |
| EHD1 | IFI44 | 0.80200687 |
| SORT1 | ZNF281 | 0.80199817 |
| DNMT1 | EPHB1 | 0.80199237 |
| CD44 | IFI44 | 0.80196048 |
| WDFY3 | RSAD2 | 0.80194888 |
| NUP205 | BLVRA | 0.80194888 |
| ALOX5AP | CCL8 | 0.80193148 |
| SORT1 | PRKAR2A | 0.80192859 |
| SMARCD3 | STAT5B | 0.80192859 |
| HERC6 | CCL8 | 0.80191989 |
| JAK3 | IRAK3 | 0.80191119 |
| ZDHHC3 | STAT5B | 0.8018706 |
| EHD1 | ENTPD7 | 0.8018677 |
| SORT1 | STAT5B | 0.8018677 |
| IFIT3 | FCER1A | 0.801859 |
| TARBP1 | CCL8 | 0.8018561 |
| PROS1 | IFI44 | 0.80183871 |
| BCL6 | EPHB1 | 0.80183291 |
| IL4R | BLVRA | 0.80177492 |
| WDFY3 | ISG20 | 0.80177203 |
| PHTF1 | BLVRA | 0.80173434 |
| GAS7 | PROS1 | 0.80172274 |
| CD44 | NARF | 0.80171984 |
| ZDHHC3 | OAS1 | 0.80170824 |
| SLC1A3 | IFI27 | 0.80170534 |
| STAT5B | OAS2 | 0.80166765 |
| GZMB | BLVRA | 0.80166185 |
| GRB10 | PROS1 | 0.80165606 |
| CD44 | PRKAR2A | 0.80163576 |
| NDST2 | IFI44L | 0.80162126 |
| VNN1 | IFI44L | 0.80160097 |
| MICAL1 | EPHB1 | 0.80159807 |
| HDAC4 | IFI44 | 0.80158357 |
| OAS2 | CD86 | 0.80156908 |
| ALOX5AP | GZMB | 0.80156328 |
| ALOX5AP | IFIT3 | 0.80155748 |
| PRKAR2A | ICAM2 | 0.80154009 |
| NARF | TARBP1 | 0.8014821 |
| DAAM2 | DRAP1 | 0.8014705 |
| ACAA1 | ST3GAL5 | 0.8014676 |
| BMX | CAPN2 | 0.80141252 |
| MICAL1 | OAS1 | 0.80140672 |
| ALOX5AP | CAPN2 | 0.80133424 |
| PGD | OAS2 | 0.80132844 |
| PHTF1 | TLR7 | 0.80131684 |
| NFKBIA | GZMB | 0.80130525 |
| ICAM2 | DRAP1 | 0.80129945 |
| BMX | EPHB1 | 0.80127915 |
| PROS1 | IFIT3 | 0.80126755 |
| VNN1 | RSAD2 | 0.80125886 |
| NFKBIA | BLVRA | 0.80124726 |
| SORT1 | VSIG4 | 0.80122697 |
| RSAD2 | CD86 | 0.80120667 |
| WDFY3 | TLR7 | 0.80118928 |
| BCL6 | IFIT2 | 0.80118638 |
| OAS1 | ITGA4 | 0.80118348 |
| ISG20 | HESX1 | 0.80113709 |
| CA4 | EBI3 | 0.80112259 |
| TMEM123 | RIN2 | 0.80111389 |
| EHD1 | SMPDL3A | 0.8010994 |
| TARBP1 | IFI44 | 0.8010994 |
| IFIT3 | IL2RB | 0.8010965 |
| ALOX5AP | IFI44L | 0.8010849 |
| BMX | PTPRO | 0.8010849 |
| CDK5RAP2 | BLVRA | 0.801082 |
| SMPDL3A | IFI44 | 0.8010762 |
| SMPDL3A | SAMD9 | 0.80106171 |
| IL1R2 | OAS1 | 0.80104141 |
| SOD2 | ST3GAL5 | 0.80104141 |
| CR1 | PTPRO | 0.80103851 |
| HDAC4 | CAPN2 | 0.80102402 |
| TARBP1 | IFI27 | 0.80099792 |
| ZDHHC3 | RABGAP1L | 0.80099213 |
| ISG20 | IL2RB | 0.80099213 |
| LY6E | MX1 | 0.80098343 |
| GAS7 | CCL8 | 0.80097473 |
| TMEM123 | DRAP1 | 0.80092834 |
| TMEM123 | ITGA4 | 0.80091964 |
| ATP9A | ACAA1 | 0.80089645 |
| ALPL | LY6E | 0.80087905 |
| GRB10 | ZNF281 | 0.80087326 |
| STAT5B | TLR7 | 0.80087326 |
| GRB10 | GZMB | 0.80084136 |
| HERC6 | OAS1 | 0.80080947 |
| ATP9A | RABGAP1L | 0.80076888 |
| GAS7 | PRKAR2A | 0.80076308 |
| IFI44L | RIN2 | 0.80075729 |
| NFKBIA | NUP205 | 0.80075729 |
| SORT1 | ACAA1 | 0.80074569 |
| MKNK1 | OAS2 | 0.80073119 |
| IFI27 | HESX1 | 0.80073119 |
| WDFY3 | TARBP1 | 0.8007196 |
| ZNF281 | EPHB1 | 0.8006935 |
| BCL6 | SAMD9 | 0.80067611 |
| FLOT2 | BLVRA | 0.80064711 |
| SORT1 | NDST2 | 0.80062972 |
| PADI2 | IFI44 | 0.80062682 |
| FKBP5 | RIN2 | 0.80053984 |
| RSAD2 | HESX1 | 0.80052824 |
| SUCLG2 | IFI44 | 0.80047026 |
| IL4R | EPHB1 | 0.80047026 |
| ZDHHC3 | PADI2 | 0.80045866 |
| IL1R2 | IFI44L | 0.80044707 |
| ACAA1 | OAS1 | 0.80041807 |
| IL4R | SUCLG2 | 0.80040358 |
| SORT1 | GRB10 | 0.80039778 |
| GRB10 | IFIT5 | 0.80038618 |
| DNMT1 | PTPRO | 0.80036299 |
| DRAP1 | HESX1 | 0.80035719 |
| VNN1 | TMEM123 | 0.80035429 |
| NFKBIA | TLR7 | 0.80034269 |
| IL1R2 | IFI44 | 0.8003253 |
| DACH1 | BLVRA | 0.8003021 |
| HERC6 | RSAD2 | 0.80025571 |
| SORT1 | SOD2 | 0.80025282 |
| SMARCD3 | GZMB | 0.80023542 |
| SMARCD3 | DACH1 | 0.80023542 |
| HDAC4 | TARBP1 | 0.80022672 |
| SMARCD3 | ICAM1 | 0.80019483 |
| IL2RB | IFIT5 | 0.80018323 |
| DAAM2 | OAS2 | 0.80016874 |
| NDST2 | RSAD2 | 0.80016294 |
| TARBP1 | IFIT3 | 0.80012815 |
| IFI44L | ITGA4 | 0.80011945 |
| STAT5B | RABGAP1L | 0.80011655 |
| CD44 | ST3GAL5 | 0.80008466 |
| GAS7 | RABGAP1L | 0.80008176 |
| VSIG4 | SUCLG2 | 0.80007886 |
| IL1R2 | TMEM123 | 0.80005856 |
| MAPK14 | CAPN2 | 0.80004697 |
| SMPDL3A | IFI27 | 0.80002087 |
| PRF1 | PTPRO | 0.80001798 |
| ZDHHC3 | SAMD9 | 0.80001508 |
| FLOT2 | OAS1 | 0.80566863 |
| SMARCD3 | RSAD2 | 0.80562224 |
| ALCAM | MX1 | 0.80560194 |
| GRB10 | TLR7 | 0.80559904 |
| SMARCD3 | EBI3 | 0.80557585 |
| HDAC4 | SAMD9 | 0.80557295 |
| RSAD2 | PRF1 | 0.80556135 |
| PHTF1 | RSAD2 | 0.80554686 |
| GRB10 | IFI27 | 0.80553236 |
| PHTF1 | IFI44 | 0.80551207 |
| OAS1 | DNMT1 | 0.80547728 |
| DYSF | BLVRA | 0.80541929 |
| EBI3 | IFI27 | 0.80540189 |
| IRAK3 | IFIT2 | 0.80537 |
| NDST2 | CD86 | 0.80532362 |
| ZDHHC3 | OAS2 | 0.80531782 |
| ALPL | HERC6 | 0.80530912 |
| SUCLG2 | TLR7 | 0.80530622 |
| JAK3 | RSAD2 | 0.80530332 |
| ICAM1 | EBI3 | 0.80529462 |
| EHD1 | BMX | 0.80526563 |
| CD44 | DNMT1 | 0.80526273 |
| JAK3 | PADI4 | 0.80523954 |
| MKNK1 | BLVRA | 0.80521634 |
| MAPK14 | IFI44L | 0.80508588 |
| EBI3 | IFIT5 | 0.80505108 |
| ATP9A | LY86 | 0.80502789 |
| ZNF281 | SUCLG2 | 0.80501339 |
| CR1 | LY86 | 0.8049989 |
| RSAD2 | DNMT1 | 0.8049757 |
| NDST2 | VSIG4 | 0.80496701 |
| PGD | OAS1 | 0.80495541 |
| EBI3 | GNLY | 0.80495541 |
| WDFY3 | EBI3 | 0.80495251 |
| CR1 | EPHB1 | 0.80493801 |
| HDAC4 | DRAP1 | 0.80492642 |
| DYSF | IFI44L | 0.80491772 |
| PRF1 | BLVRA | 0.80491482 |
| EHD1 | MAPK14 | 0.80488873 |
| TMEM123 | EPHB1 | 0.80481914 |
| ATP9A | PTPRO | 0.80479885 |
| OAS2 | DNMT1 | 0.80478725 |
| PHTF1 | IFI44L | 0.80477276 |
| EHD1 | ALOX5AP | 0.80474666 |
| PADI2 | IFI44L | 0.80474666 |
| ATP9A | SUCLG2 | 0.80474666 |
| MICAL1 | CD86 | 0.80473217 |
| HERC6 | IFI27 | 0.80471477 |
| SUCLG2 | CCL8 | 0.80468288 |
| SORL1 | SUCLG2 | 0.80467998 |
| CR1 | DRAP1 | 0.80466258 |
| GZMB | CD86 | 0.80465969 |
| SORT1 | GAS7 | 0.80464809 |
| TARBP1 | GZMB | 0.80463069 |
| BCL6 | IFI44L | 0.8046162 |
| CD44 | SOD2 | 0.804593 |
| JAK3 | IFI44 | 0.80457851 |
| EHD1 | FCER1A | 0.80457561 |
| FCER1A | SAMD9 | 0.80455241 |
| IL4R | SAMD9 | 0.80454661 |
| MICAL1 | ST3GAL5 | 0.80452632 |
| MICAL1 | OAS2 | 0.80451762 |
| IFI44L | PRF1 | 0.80450602 |
| SORT1 | GZMB | 0.80449443 |
| CD44 | RABGAP1L | 0.80449153 |
| CPD | SOD2 | 0.80448863 |
| ALCAM | SUCLG2 | 0.80448573 |
| SUCLG2 | PTPRO | 0.80445964 |
| JAK3 | SORL1 | 0.80441905 |
| NFKBIA | CCL8 | 0.80441325 |
| MX2 | FCER1A | 0.80440165 |
| RIN2 | HESX1 | 0.80439875 |
| SUCLG2 | IFIT3 | 0.80439585 |
| TMEM123 | BLVRA | 0.80438136 |
| DRAP1 | GNLY | 0.80436106 |
| OAS2 | PRF1 | 0.80432627 |
| CPD | NFKBIA | 0.80431757 |
| DYSF | SAMD9 | 0.80429728 |
| IRAK3 | RABGAP1L | 0.80426829 |
| FLOT2 | OAS2 | 0.80425089 |
| VSIG4 | DRAP1 | 0.80423929 |
| VSIG4 | CD86 | 0.8042219 |
| STAT5B | SUCLG2 | 0.8042219 |
| CD44 | BMX | 0.804219 |
| JAK3 | SOD2 | 0.8042132 |
| JAK3 | ALCAM | 0.8041987 |
| IFIT1 | MX1 | 0.80417551 |
| LY86 | SAMD9 | 0.80415232 |
| PHTF1 | ISG20 | 0.80414362 |
| RSAD2 | ITGA4 | 0.80413782 |
| EHD1 | MKNK1 | 0.80594696 |
| CPD | IFI44L | 0.80594116 |
| MX2 | LY86 | 0.80589767 |
| CR1 | BLVRA | 0.80588317 |
| SORL1 | CAPN2 | 0.80587157 |
| GNLY | BLVRA | 0.80587157 |
| LY6E | BLVRA | 0.80586868 |
| IFI44L | SUCLG2 | 0.80586578 |
| SORT1 | ST3GAL5 | 0.80586288 |
| TARBP1 | IFI44L | 0.80585998 |
| JAK3 | NARF | 0.80585418 |
| GAS7 | TLR7 | 0.80584838 |
| ENTPD7 | TMEM123 | 0.80583968 |
| NUP205 | CD86 | 0.80582519 |
| RSAD2 | GZMB | 0.8057875 |
| PROS1 | IFI44L | 0.8057759 |
| SOD2 | CD86 | 0.8057556 |
| VNN1 | RIN2 | 0.80572661 |
| CPD | STAT5B | 0.80570922 |
| ATP9A | TARBP1 | 0.80570632 |
| IL4R | TARBP1 | 0.80568602 |
| NDST2 | ATP9A | 0.80608032 |
| TARBP1 | ICAM2 | 0.80607742 |
| PADI2 | RSAD2 | 0.80605423 |
| SMARCD3 | IFI44L | 0.80601074 |
| MAPK14 | IFI44 | 0.80595565 |

### REFERENCES

1 Andres-Terre, M. et al. Integrated, Multi-cohort Analysis Identifies Conserved Transcriptional Signatures across Multiple Respiratory Viruses. Immunity 43, 1199-1211, doi:10.1016/j.immuni.2015.11.003 (2015).
2 Sweeney, T. E., Wong, H. R. & Khatri, P. Robust classification of bacterial and viral infections via integrated host gene expression diagnostics. Sci Transl Med 8, 346ra391, doi:10.1126/scitranslmed.aaf7165 (2016).
3 Edgar, R., Domrachev, M. & Lash, A. E. Gene Expression Omnibus: NCBI gene expression and hybridization array data repository. Nucleic Acids Res 30, 207-210 (2002).
4 Kolesnikov, N. et al. ArrayExpress update—simplifying data submissions. Nucleic Acids Res 43, D1113-1116, doi:10.1093/nar/gku1057 (2015).
5 Sweeney, T. E., Shidham, A., Wong, H. R. & Khatri, P. A comprehensive time-course-based multicohort analysis of sepsis and sterile inflammation reveals a robust diagnostic gene set. Sci Transl Med 7, 287ra271, doi:10.1126/scitranslmed.aaa5993 (2015).
6 Ardura, M. I. et al. Enhanced monocyte response and decreased central memory T cells in children with invasive Staphylococcus aureus infections. PLoS One 4, e5446, doi:10.1371/journal.pone.0005446 (2009).
7 Parnell, G. et al. Aberrant cell cycle and apoptotic changes characterise severe influenza A infection—a meta-analysis of genomic signatures in circulating leukocytes. PLoS One 6, e17186, doi:10.1371/journal.pone.0017186 (2011).
8 Parnell, G. P. et al. A distinct influenza infection signature in the blood transcriptome of patients with severe community-acquired pneumonia. Crit Care 16, R157, doi:10.1186/cc11477 (2012).
9 Banchereau, R. et al. Host immune transcriptional profiles reflect the variability in clinical disease manifestations in patients with Staphylococcus aureus infections. PLoS One 7, e34390, doi:10.1371/journal.pone.0034390 (2012).
10 Berdal, J. E. et al. Excessive innate immune response and mutant D222G/N in severe A (H1N1) pandemic influenza. J Infect 63, 308-316, doi:10.1016/j.jinf.2011.07.004 (2011).
11 de Steenhuijsen Piters, W. A. et al. Nasopharyngeal Microbiota, Host Transcriptome, and Disease Severity in Children with Respiratory Syncytial Virus Infection. Am J Respir Crit Care Med 194, 1104-1115, doi:10.1164/rccm.201602-0220OC (2016).
12 Heinonen, S. et al. Rhinovirus Detection in Symptomatic and Asymptomatic Children: Value of Host Transcriptome Analysis. Am J Respir Crit Care Med 193, 772-782, doi:10.1164/rccm.201504-0749OC (2016).
13 Herberg, J. A. et al. Transcriptomic profiling in childhood H1N1/09 influenza reveals reduced expression of protein synthesis genes. J Infect Dis 208, 1664-1668, doi:10.1093/infdis/jit348 (2013).
14 Ioannidis, I. et al. Plasticity and virus specificity of the airway epithelial cell immune response during respiratory virus infection. J Virol 86, 5422-5436, doi:10.1128/JVI.06757-11 (2012).
15 Jong, V. L. et al. Transcriptome assists prognosis of disease severity in respiratory syncytial virus infected infants. Sci Rep 6, 36603, doi:10.1038/srep36603 (2016).
16 Lindow, J. C. et al. Cathelicidin Insufficiency in Patients with Fatal Leptospirosis. PLoS Pathog 12, e1005943, doi:10.1371/journal.ppat.1005943 (2016).
17 Liu, T. Y. et al. An individualized predictor of health and disease using paired reference and target samples. BMC Bioinformatics 17, 47, doi:10.1186/s12859-016-0889-9 (2016).
18 Mejias, A. et al. Whole blood gene expression profiles to assess pathogenesis and disease severity in infants with respiratory syncytial virus infection. PLoS Med 10, e1001549, doi:10.1371/journal.pmed.1001549 (2013).
19 Rodriguez-Fernandez, R. et al. Respiratory Syncytial Virus Genotypes, Host Immune Profiles, and Disease Severity in Young Children Hospitalized With Bronchiolitis. J Infect Dis 217, 24-34, doi:10.1093/infdis/jix543 (2017).
20 Suarez, N. M. et al. Superiority of transcriptional profiling over procalcitonin for distinguishing bacterial from viral lower respiratory tract infections in hospitalized adults. J Infect Dis 212, 213-222, doi:10.1093/infdis/jiv047 (2015).
21 Tang, B. M. et al. A novel immune biomarker IFI27 discriminates between influenza and bacteria in patients with suspected respiratory infection. Eur Respir J 49, doi:10.1183/13993003.02098-2016 (2017).
22 Zaas, A. K. et al. Gene expression signatures diagnose influenza and other symptomatic respiratory viral infections in humans. Cell Host Microbe 6, 207-217, doi:10.1016/j.chom.2009.07.006 (2009).
23 Tsalik, E. L. et al. Host gene expression classifiers diagnose acute respiratory illness etiology. Sci Transl Med 8, 322ra311, doi:10.1126/scitranslmed.aad6873 (2016).
24 Sweeney, T. E., Braviak, L., Tato, C. M. & Khatri, P. Genome-wide expression for diagnosis of pulmonary tuberculosis: a multicohort analysis. Lancet Respir Med 4, 213-224, doi:10.1016/52213-2600(16)00048-5 (2016).
25 Khatri, P. et al. A common rejection module (CRM) for acute rejection across multiple organs identifies novel therapeutics for organ transplantation. J Exp Med 210, 2205-2221, doi:10.1084/jem.20122709 (2013).
26 Johnson, W. E., Fau, L. C. & Rabinovic, A. Adjusting batch effects in microarray expression data using empirical Bayes methods. Biostatistics (2006).
27 Tusher, V. G., Tibshirani, R. & Chu, G. Significance analysis of microarrays applied to the ionizing radiation response. Proc Natl Acad Sci USA 98, 5116-5121, doi:10.1073/pnas.091062498 (2001).
28 Benjamini, Y. & Hochberg, Y. Controlling the False Discovery Rate: A Practical and Powerful Approach to Multiple Testing. J R Stat Soc Series B Stat Methodol 57, 289-300 (1995).

That which is claimed is:

1. A method of analyzing a sample, the method comprising:
(a) obtaining a sample of RNA from a subject; and
(b) measuring the amount of RNA transcripts encoded by JUP, SUCLG2, IFI27, FCER1A, HESX1, SMARCD3, ICAM1, and EBI3 in the sample, to produce gene expression data.

2. The method of claim 1, wherein the measuring step is done by RT-PCR.

3. The method of claim 1, wherein the measuring step is done using a quantitative isothermal amplification method.

4. The method of claim 1, wherein the measuring step is done by sequencing.

5. The method of claim 1, wherein the measuring step is done by labeling the RNA or cDNA made from the same and hybridizing the labeled RNA or cDNA to a support.

6. The method of any prior claim, wherein the sample comprises RNA isolated from whole blood, white blood cells, neutrophils, peripheral blood mononuclear cells (PBMCs), or buffy coat.

7. The method of claims 1-6, further comprising:
(c) based on the gene expression data, providing a report indicating whether the subject has a viral infection or a bacterial infection, wherein:
(i) increased JUP, SUCLG2, IFI27, FCER1A, HESX1 expression indicates that the subject has a viral infection; and
(ii) increased SMARCD3, ICAM1, EBI3 indicates that the subject has a bacterial infection.

8. A method for treating a subject, comprising:
(a) receiving a report indicating whether the subject has a viral infection or a bacterial infection, wherein the report is based on the gene expression data obtained by measuring the amount of RNA transcripts encoded by JUP, SUCLG2, IFI27, FCER1A, HESX1, SMARCD3, ICAM1, and EBI3, and
(b) identifying the patient as having increased JUP, SUCLG2, IFI27, and FCER1A, and HESX1 expression; and
treating the subject with anti-viral therapy; or
(c) identifying the patient as having increased SMARCD3, ICAM1, EBI3 expression; and
treating the subject with an anti-bacterial therapy.

9. The method of claim 8, wherein step (b) comprises administering an anti-viral agent to the subject.

10. The method of claim 8, wherein step (c) comprises administering an antibiotic to the subject.

11. A kit comprising reagents for measuring the amount of RNA transcripts encoded by JUP, SUCLG2, IFI27, FCER1A, HESX1, SMARCD3, ICAM1, and EBI3.

12. The kit of claim 11, wherein the reagents comprise, for each RNA transcript, a sequence-specific oligonucleotide that hybridizes to the transcript.

13. The kit of claim 12, wherein sequence-specific oligonucleotide is biotinylated and/or labeled with an optically-detectable moiety.

14. The kit of claim 11, wherein the reagents comprises, for each RNA transcript, a pair of PCR primers that amplify a sequence from the RNA transcript, or cDNA made from the same.

15. The kit of claim 11, wherein the reagents comprise multiple reaction vessels, each comprising at least one sequence-specific isothermal amplification primer that hybridizes to the transcript, or cDNA made from the same.
